(19) United States
(12) Patent Application Publication
(10) Pub. No.: US 2025/0160698 A1
(43) Pub. Date: May 22, 2025

(54) SYSTEMS AND METHODS FOR IMPROVED IN VIVO ANALYTE SENSOR FUNCTION

(71) Applicant: Abbott Diabetes Care Inc., Alameda, CA (US)

(72) Inventors: Benjamin J. FELDMAN, Berkeley, CA (US); Zenghe LIU, Alameda, CA (US)

(73) Assignee: Abbott Diabetes Care Inc., Alameda, CA (US)

(21) Appl. No.: 19/031,676

(22) Filed: Jan. 18, 2025

Related U.S. Application Data
(63) Continuation of application No. 16/815,791, filed on Mar. 11, 2020.
Application No. 16/815,791 is a continuation of application No. 13/188,757, filed on Jul. 22, 2011, now abandoned.
(60) Provisional application No. 61/366,811, filed on Jul. 22, 2010.

(51) Int. Cl. A61B 5/145; A61B 5/1486; C12Q 1/00; G01N 27/327

(52) U.S. Cl. CPC A61B 5/14532; A61B 5/1486; C12Q 1/005; C12Q 1/006; G01N 27/3271; G01N 27/3272; C12Q 1/001; G01N 27/327; Y10S 424/81; Y10S 530/868

(57) ABSTRACT

An analyte sensor comprising: an insertion tip configured for insertion below a tissue of a user, the insertion tip comprising: a working electrode comprising a sensing layer disposed thereon and a membrane layer disposed at least partially over the sensing layer; a substrate; and a counter electrode. Wherein a polymer layer comprising at least an immunosuppressant is disposed on an exterior surface of the working electrode, the substrate, or the counter electrode, the polymer layer being separate from the sensing layer and the membrane layer.

### CROSS-REFERENCE TO RELATED APPLICATION

This patent application claims the benefit of priority under 35 U.S.C. § 119 (e) to U.S. Provisional Patent Application No. 61/366,811, filed Jul. 22, 2010, the disclosure of which is hereby incorporated by reference in its entirety.

### INTRODUCTION

In many instances it is desirable or necessary to regularly monitor the concentration of particular constituents in a fluid. A number of systems are available that analyze the constituents of bodily fluids such as blood, urine and saliva. Examples of such systems conveniently monitor the level of particular medically significant fluid constituents, such as, for example, cholesterol, ketones, vitamins, proteins, and various metabolites or blood sugars, such as glucose. Diagnosis and management of patients suffering from diabetes mellitus, a disorder of the pancreas where insufficient production of insulin prevents normal regulation of blood sugar levels, requires carefully monitoring of blood glucose levels on a daily basis. A number of systems that allow individuals to easily monitor their blood glucose are currently available. Such systems include electrochemical biosensors, including those that comprise a glucose sensor that is adapted for insertion into a subcutaneous site within the body for the continuous monitoring of glucose levels in bodily fluid of the subcutaneous site (see for example, U.S. Pat. No. 6,175,752 to Say et al).

A person may obtain a blood sample by withdrawing blood from a blood source in his or her body, such as a vein, using a needle and syringe, for example, or by lancing a portion of his or her skin, using a lancing device, for example, to make blood available external to the skin, to obtain the necessary sample volume for in vitro testing. The person may then apply the fresh blood sample to a test strip, whereupon suitable detection methods, such as calorimetric, electrochemical, or photometric detection methods, for example, may be used to determine the person's actual blood glucose level. The foregoing procedure provides a blood glucose concentration for a particular or discrete point in time, and thus, must be repeated periodically, in order to monitor blood glucose over a longer period.

In addition to the discrete or periodic, or in vitro, blood glucose-monitoring systems described above, at least partially implantable, or in vivo, blood glucose-monitoring systems, which are constructed to provide continuous in vivo measurement of an individual's blood glucose concentration, have been described and developed.

Such analyte monitoring devices are constructed to provide for continuous or automatic monitoring of analytes, such as glucose, in the blood stream or interstitial fluid. Such devices include electrochemical sensors, at least a portion of which are operably positioned in a blood vessel or in the subcutaneous tissue of a user.

While continuous glucose monitoring is desirable, there are several challenges associated with optimizing the biosensors constructed for in vivo use. Accordingly, further development of manufacturing techniques and methods, as well as analyte-monitoring devices, systems, or kits employing the same, is desirable.

### SUMMARY

Embodiments of the present disclosure relate to systems for improving the performance of one or more components of a sensor, such as an in vivo analyte sensor, including, for example, continuous and/or automatic in vivo analyte sensors, by detecting inflammation at an insertion site and adjusting the signal of the sensor, adjusting the display of the signal (e.g., inactivation of display), or indicating administration of an anti-inflammatory agent, such as an interleukin 1 receptor antagonist. Embodiments of the present disclosure also relate to analyte determining methods and devices (e.g., electrochemical analyte monitoring systems) that have improved signal response and stability by inclusion of one or more of a clot activator and/or an immunosuppressant proximate to a working electrode of an in vivo analyte sensor. Also provided are systems and methods of using the, for example electrochemical, analyte sensors in analyte monitoring.

These and other objects, advantages, and features of embodiments of the present disclosure will become apparent to those persons skilled in the art upon reading the details as more fully described herein.

### INCORPORATION BY REFERENCE

The following patents, applications and/or publications are incorporated herein by reference for all purposes: U.S. Pat. Nos. 7,041,468; 5,356,786; 6,175,752; 6,560,471; 5,262,035; 6,881,551; 6,121,009; 7,167,818; 6,270,455; 6,161,095; 5,918,603; 6,144,837; 5,601,435; 5,822,715; 5,899,855; 6,071,391; 6,120,676; 6,143,164; 6,299,757; 6,338,790; 6,377,894; 6,600,997; 6,773,671; 6,514,460; 6,592,745; 5,628,890; 5,820,551; 6,736,957; 4,545,382; 4,711,245; 5,509,410; 6,540,891; 6,730,200; 6,764,581; 6,299,757; 6,461,496; 6,503,381; 6,591,125; 6,616,819; 6,618,934; 6,676,816; 6,749,740; 6,893,545; 6,942,518; 6,514,718; 5,264,014; 5,262,305; 5,320,715; 5,593,852; 6,746,582; 6,284,478; 7,299,082; U.S. Patent Application No. 61/149,639, entitled “Compact On-Body Physiological Monitoring Device and Methods Thereof”, U.S. patent application Ser. No. 11/461,725, filed Aug. 1, 2006, entitled “Analyte Sensors and Methods”; U.S. patent application Ser. No. 12/495,709, filed Jun. 30, 2009, entitled “Extruded Electrode Structures and Methods of Using Same”; U.S. Patent Application Publication No. US2004/0186365; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; U.S. Patent Application Publication No. 2007/0068807; US patent Application Publication No. 2010/0198034; and U.S. provisional application No. 61/149,639 titled “Compact On-Body Physiological Monitoring Device and Methods Thereof”, the disclosures of each of which are incorporated herein by reference in their entirety.

### BRIEF DESCRIPTION OF THE DRAWINGS

Aspects of embodiments of the present disclosure are best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures.

FIGS. 1A-H show Continuous Glucose Monitoring (CGM) in Normal C57BL/6 Mice over a 7 day time period. FIGS. 1A-H are representative of CGM in C57BL/6 mice for 7 days post sensor implantation. FIG. 1E represents the magnified view of FIG. 1A, FIG. 1F represents the magnified view of FIG. 1B, FIG. 1G represents the magnified view of FIG. 1C, and FIG. 1H represents the magnified view of FIG. 1D. Sensor output is expressed as CGS output (nA) and is represented by the blue lines. Blood glucose levels are represented by red diamonds.

FIGS. 2A-H show continuous glucose monitoring (CGM) in IL-1RN-KO over a 7-day time period. FIGS. 2A-H are representative of CGM in IL-1RN knockout mice (IL-1RN-KN). FIG. 2E represents the magnified view of FIG. 2A, FIG. 2F represents the magnified view of FIG. 2B, FIG. 2G represents the magnified view of FIG. 2C and FIG. 2H represents the magnified view of FIG. 2D. Sensor output is expressed as CGS output (nA) and is represented by the blue lines. Blood glucose levels are represented by red diamonds.

FIGS. 3A-H show continuous glucose monitoring (CGM) in IL-1RN-EO Mice over a 7-day time period. FIGS. 3A-H are representative of CGM in IL-1RN overexpressor mice (IL-1RN-OE). FIG. 3E represents the magnified view of FIG. 3A, FIG. 3F represents the magnified view of FIG. 3B, FIG. 3G represents the magnified view of FIG. 3C and FIG. 3H represents the magnified view of FIG. 3D. Sensor output is expressed as CGS output (nA) and is represented by the blue lines. Blood glucose levels are represented by red diamonds.

FIG. 4 shows tissue reactions induced at sites of Glucose Sensor Implantation in C57B/6, IL-1RN-KO and IL-1RN-EO mice over a 7-day period. Histopathologic analysis of tissue from sensor implantation sites in C57BL/6 (Panels A-C), IL-1RN-KO (Panels D-F), and IL-1RN-OE (Panels G-I) mice was evaluated using standard H&E staining techniques. Location of the sensor in the tissue is designated by the asterisk symbol (*). In H&E sections the residual sensor coating appears as a black layer associated with the asterisk symbol.

FIG. 5 shows evaluation of fibrotic tissue response to implanted glucose sensors over a 7-day period. To evaluate the collagen distribution in tissue response associated with various segments of the glucose sensor implanted in the mice for up to 7 days, mouse tissue from the sensor sites was obtained and processed for trichrome staining (collagen stains blue in the sections). FIG. 5 shows the histopathologic analysis of tissue from sensor implantation sites in C57BL/6 (Panels A-C), IL-1RN-KO (Panels D-F), and IL-1RN-OE (Panels G-I) mice. In the Masson Trichrome sections, the residual sensor coating appears as an orange layer associated with the asterisk symbol (*).

FIGS. 6A-C show a hypothetical model of IL-1B and IL-1RN tissue and sensor interactions at sites of glucose sensor implantation in normal tissue. The model outlines the various possible IL-1 related pathways that are involved in controlling tissue reactions at sites of glucose sensor tissue reactions, as well as glucose sensor function in vivo in normal mice (FIG. 6A), IL-1RN KO (FIG. 6B), and IL-1OE mice (FIG. 6C). The symbols and abbreviation used in this figure include: M1 macrophages (red cells), M2 macrophages (green cells), IL-1B (red triangles), IL-1RN (green triangles), pro-inflammatory and pro-fibrotic factors (red stars), anti-inflammatory and anti-fibrosis factors (green circles and ovals), leukocyte chemotactic factors (LCF), vasopermeability factors (VP). Red arrows down equate to loss of sensor function and green arrows up equate to extended sensor function and lifespan.

FIG. 7 shows a block diagram of an embodiment of an analyte monitoring system according to embodiments of the present disclosure.

FIG. 8 shows a block diagram of an embodiment of a data processing unit of the analyte monitoring system shown in FIG. 7.

FIG. 9 shows a block diagram of an embodiment of the primary receiver unit of the analyte monitoring system of FIG. 7.

FIG. 10 shows a schematic diagram of an embodiment of an analyte sensor according to the embodiments of the present disclosure.

### DETAILED DESCRIPTION

Before embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

Various terms are described below to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Systems and Methods Using Anti-Inflammatory Agents

Embodiments of the present disclosure relate to methods and devices for improving the signal response and/or stability of a sensor by inclusion of an anti-inflammatory agent disposed proximate to a working electrode of the sensor, such as in vivo analyte sensor, including, for example, continuous and/or automatic in vivo analyte sensors. For example, embodiments of the present disclosure provide for inclusion of an anti-inflammatory agent, resulting in an increase in the stability of the signal from the sensor over time and/or an increase in signal response. In certain embodiments, inclusion of the anti-inflammatory agent results in an increase in the stability of the signal from the sensor and/or an increase in signal response following insertion of in vivo biosensor in a user. In some instances, inclusion of the anti-inflammatory agent results in an increase in the stability of the signal from the sensor over time, thus increasing the lifespan of the sensor. Also provided are systems and methods of using the analyte sensors in analyte monitoring.

Embodiments of the present disclosure are based on the discovery that the addition of an anti-inflammatory agent to in vivo biosensors improves signal response and stability of the sensor. Biocompatible layers of embodiments of the present disclosure can include anti-inflammatory agents, e.g., compounds or compositions that decrease the occurrence and/or severity of inflammation in the tissues surrounding the site of the in vivo biosensor insertion in the user. In some cases, the anti-inflammatory agent is included in a membrane formulation disposed over the sensor. During use, the anti-inflammatory agent may diffuse out of the membrane formulation into the surrounding tissues. In some instances, the anti-inflammatory agent is applied to an exterior surface of the sensor, such that the anti-inflammatory agent is readily available on the surface of the sensor following insertion of the sensor in the user.

During in vivo use of the subject analyte sensors, a portion of the analyte sensor is inserted beneath a skin surface of a user. Following insertion of the analyte sensor, there may be a transient reduction in signal from the sensor. Without being limited to any particular theory, inflammation in the tissues surrounding the sensor insertion site may result in a decrease in signal from the sensor. This may result in variable data quality before the signal from the sensor stabilizes, resulting in a so-called Early Signal Attenuation (ESA) effect.

Embodiments of the present disclosure provide for increased signal response and/or stability by decreasing the ESA effect. The result may be a reduction, and in some cases, complete elimination of the ESA effect. As such, embodiments that include the anti-inflammatory agent may provide for increased signal response and/or stability, such that substantially no ESA occurs following subcutaneous insertion of the analyte sensor. For example, as compared to sensors that do not include an anti-inflammatory agent, sensors that include an anti-inflammatory agent may have a reduction in the ESA effect for 30 min or more following subcutaneous insertion of the analyte sensor, such as 1 hour or more, including 2 hours or more, or 4 hours or more, or 6 hours or more, or 8 hours or more, or 10 hours or more, or 12 hours or more, for instance 14 hours or more, or 18 hours or more, or 24 hours or more, including 2 days or more, or 3 days or more, or 4 days or more, or 5 days or more, or 6 days or more, or 7 days or more, or 10 days or more, or 14 days or more following subcutaneous insertion of the analyte sensor. As such, sensors that include an anti-inflammatory agent may allow stable signal to be detected within a certain time period following subcutaneous insertion of the analyte sensor, such as 24 hours or less, or 18 hours or less, or 12 hours or less, or 8 hours or less, or 6 hours or less, or 5 hours or less, or 4 hours or less, or 3 hours or less, or 2 hours or less, or 1 hour or less, including 45 minutes or less, such as 30 minutes or less, for example, 15 minutes or less, or 10 minutes or less, or 5 minutes or less, or 3 minutes or less, or 2 minutes or less, or 1 minute or less following subcutaneous insertion of the analyte sensor. In some instances, sensors that include an anti-inflammatory agent may allow stable signal to be detected immediately following subcutaneous insertion of the analyte sensor.

In certain embodiments of the present disclosure, inclusion of the anti-inflammatory agent results in an increase in the accuracy of the analyte measurements from the sensor. For example, inclusion of the anti-inflammatory agent may result in better correlation between the analyte concentration as determined by the in vivo analyte monitoring device (e.g., based on signals detected from the analyte sensor) and a reference analyte concentration. In certain instances, inclusion of the anti-inflammatory agent results in analyte concentrations as determined by the signals detected from the analyte sensor that are within 50% of a reference value, such as within 40% of the reference value, including within 30% of the reference value, or within 20% of the reference value, or within 10% of the reference value, or within 5% of the reference value, or within 2% of the reference value, or within 1% of the reference value. In some cases, the analyte sensors maintains its accuracy (e.g., is within a threshold percentage of a reference value, as described above) for 75% or more of the time during use, such as 80% or more, or 90% or more, including 95% or more, or 97% or more, or 99% or more of the time during use. As an alternative measure of accuracy, in some cases, inclusion of the anti-inflammatory agent results in analyte concentrations as determined by the signals detected from the analyte sensor that are within Zone A of the Clarke Error Grid Analysis. For example, inclusion of the anti-inflammatory agent may result in analyte concentrations as determined by the signals detected from the analyte sensor that are within Zone A of the Clarke Error Grid Analysis for 75% or more of the time during use, such as 80% or more, or 90% or more, including 95% or more, or 97% or more, or 99% or more of the time during use. In certain instances, inclusion of the anti-inflammatory agent results in analyte concentrations as determined by the signals detected from the analyte sensor that are within Zone A or Zone B of the Clarke Error Grid Analysis. For example, inclusion of the anti-inflammatory agent may result in analyte concentrations as determined by the signals detected from the analyte sensor that are within Zone A or Zone B of the Clarke Error Grid Analysis for 75% or more of the time during use, such as 80% or more, or 90% or more, including 95% or more, or 97% or more, or 99% or more of the time during use. Further information regarding the Clarke Error Grid Analysis is found in Clarke, W. L. et al. “Evaluating Clinical Accuracy of Systems for Self-Monitoring of Blood Glucose” Diabetes Care, vol. 10, no. 5, 1987:622-628.

In certain embodiments, sensors that include an anti-inflammatory agent have a sensitivity of 0.1 nA/mM or more, or 0.5 nA/mM or more, such as 1 nA/mM or more, including 1.5 nA/mM or more, for instance 2 nA/mM or more, or 2.5 nA/mM or more, or 3 nA/mM or more, or 3.5 nA/mM or more, or 4 nA/mM or more, or 4.5 nA/mM or more, or 5 nA/mM or more. In some cases, sensors that include an anti-inflammatory agent have a sensitivity ranging from 0.1 nA/mM to 5 nA/mM, such as from 0.1 nA/mM to 4.5 nA/mM, including from 0.1 nA/mM to 4 nA/mM, or from 0.2 nA/mM to 3.5 nA/mM, or from 0.2 nA/mM to 3 nA/mM, or from 0.3 nA/mM to 2.5 nA/mM, or from 0.3 nA/mM to 2 nA/mM.

In some instances, inclusion of an anti-inflammatory agent provides for increased signal response and/or stability over the life of the sensor. The result may be an increase in the lifespan of the sensor as compared to sensors that do not include an anti-inflammatory agent. In some cases, a sensor that includes an anti-inflammatory agent as disclosed herein has an initial sensitivity. The sensor may have a sensitivity that is 90% or more of the initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. For example, the sensor may maintain 95% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In some cases, the sensor maintains 97% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In certain instances, the sensor may maintain 99% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more.

Sensors that include an anti-inflammatory agent may also have increased linearity in signal over a wide range of analyte concentrations as compared to sensors that do not include an anti-inflammatory agent. In certain embodiments, sensors that include an anti-inflammatory agent have a substantially linear signal over a range of analyte concentrations. For example, sensors that include an anti-inflammatory agent may have a substantially linear signal over a range of blood glucose concentrations, such as from 10 to 1000 mg/dL, including from 25 to 700 mg/dL, for instance from 50 to 500 mg/dL, or from 50 to 300 mg/dL.

In certain embodiments, a sensor that includes an anti-inflammatory agent as disclosed herein has an increased lifespan as compared to a sensor that does not include an anti-inflammatory agent. For example, a sensor that includes an anti-inflammatory agent may produce accurate, detectable signals for 1 day or more, such as 2 days or more, or 3 days or more, including 4 days or more, 5 days or more, 6 days or more, 7 days or more, or 10 days or more, or 14 days or more, or 3 weeks or more, or 1 month or more. Stated another way, a sensor that includes an anti-inflammatory agent may be used by the user for 1 day or more, such as 2 days or more, or 3 days or more, including 4 days or more, 5 days or more, 6 days or more, 7 days or more, or 10 days or more, or 14 days or more, or 3 weeks or more, or 1 month or more before needing to be replaced with a new sensor.

Examples of anti-inflammatory agents suitable for use with the subject devices, methods and kits include, but are not limited to, peptide or protein anti-inflammatory agents (e.g., interleukin-1 receptor antagonist (IL-1RA/IL-1RA), steroidal anti-inflammatory agents (e.g., glucocorticoids, corticosteroids, etc.), non-steroidal anti-inflammatory drugs (NSAIDs) (e.g., salicylates, propionic acid derivatives, acetic acid derivatives, enolic acid (Oxicam) derivatives, fenamic acid derivatives, and the like). In certain embodiments, the anti-inflammatory agent is interleukin-1 receptor antagonist (IL-1RA/IL-1RA). In some cases, the anti-inflammatory agent is a steroidal anti-inflammatory agent, such as, but not limited to, hydrocortisone, prednisone, prednisolone, methylprednisolone, dexamethasone, betamethasone, combinations thereof, and the like. In certain instances, the anti-inflammatory agent is a non-steroidal anti-inflammatory drug (NSAID), such as, but not limited to, a salicylate (e.g., aspirin (acetylsalicylic acid), diflunisal, salsalate, etc.), propionic acid derivatives (e.g., ibuprofen, naproxen, fenoprofen, ketoprofen, flurbiprofen, oxaprozin, etc.), acetic acid derivatives (e.g., indomethacin, sulindac, etodolac, ketorolac, nabumetone, etc.), enolic acid (Oxicam) derivatives (e.g., piroxicam, meloxicam, tenoxicam, lornoxicam, etc.), fenamic acid derivatives (e.g., mefenamic acid, meclofenamic acid, flufenamic acid, etc.), combinations thereof, and the like.

The anti-inflammatory agent may be included in any component of a sensor that is inserted into a user during use or near the point of insertion. Embodiments include, but are not limited to, sensors that include a sensing layer having an anti-inflammatory agent, sensors that include a membrane layer having an anti-inflammatory agent, and sensors that include an anti-inflammatory agent disposed on an exterior surface of the sensor. In addition, the component formulation of a sensor (e.g., the sensing layer and/or membrane layer and/or anti-inflammatory agent formulation) may be contacted to the sensor in any of a variety of suitable ways, for example, but not limited to, dip coating, spray coating, drop deposition, and the like.

Additional embodiments of a sensor that may be suitably formulated with an anti-inflammatory agent are described in U.S. Pat. Nos. 5,262,035, 5,262,305, 6,134,461, 6,143,164, 6,175,752, 6,338,790, 6,579,690, 6,605,200, 6,605,201, 6,654,625, 6,736,957, 6,746,582, 6,932,894, 7,090,756 as well as those described in U.S. patents application Ser. Nos. 11/701,138, 11/948,915, 12/625,185, 12/625,208, and 12/624,767, the disclosures of all of which are incorporated herein by reference in their entirety. Moreover, embodiments of the present disclosure may be incorporated into battery-powered or self-powered analyte sensors, in one embodiment the analyte sensor is a self-powered sensor, such as disclosed in U.S. patent application Ser. No. 12/393,921 (Publication No. 2010/0213057).

In some embodiments, the anti-inflammatory agent is formulated with a sensing layer that is disposed on a working electrode. The sensing layer may be described as the active chemical area of the biosensor. The sensing layer formulation, which can include a glucose-transducing agent, may include, for example, among other constituents, a redox mediator, such as, for example, a hydrogen peroxide or a transition metal complex, such as a ruthenium-containing complex or an osmium-containing complex, and an analyte-responsive enzyme, such as, for example, a glucose-responsive enzyme (e.g., glucose oxidase, glucose dehydrogenase, etc.) or lactate-responsive enzyme (e.g., lactate oxidase). In certain embodiments, the sensing layer includes glucose oxidase. The sensing layer may also include other optional components, such as, for example, a polymer and a bi-functional, short-chain, epoxide cross-linker, such as polyethylene glycol (PEG).

In certain instances, the analyte-responsive enzyme is distributed throughout the sensing layer. For example, the analyte-responsive enzyme may be distributed uniformly throughout the sensing layer, such that the concentration of the analyte-responsive enzyme is substantially the same throughout the sensing layer. In some cases, the sensing layer may have a homogeneous distribution of the analyte-responsive enzyme. In certain embodiments, the redox mediator is distributed throughout the sensing layer. For example, the redox mediator may be distributed uniformly throughout the sensing layer, such that the concentration of the redox mediator is substantially the same throughout the sensing layer. In some cases, the sensing layer may have a homogeneous distribution of the redox mediator. In certain embodiments, both the analyte-responsive enzyme and the redox mediator are distributed uniformly throughout the sensing layer, as described above.

Any suitable proportion of anti-inflammatory agent may be used with the sensor, where the specifics will depend on, e.g., the particular sensing layer formulation, the particular membrane formulation, the site of sensor insertion, etc. In certain embodiments, the concentration of the anti-inflammatory agent may range from 0.1% to 25% (v/v) of the total membrane layer formulation, such as from 0.5% to 10% (v/v), including from 1% to 5% (v/v), for instance from 1.5% to 3% (v/v), and the like. In certain cases, only the membrane layer includes the anti-inflammatory agent. For instance, the anti-inflammatory agent may only be included in the membrane layer and substantially excluded from any of the other layers of the sensor, such as, but not limited to, the sensing layer. In certain embodiments, the anti-inflammatory agent is applied to the exterior surface of the sensor, such as by dip coating, spray coating, drop deposition, and the like. In these embodiments, the anti-inflammatory agent formulation may include the anti-inflammatory agent in a concentration ranging from 0.1% to 25% (v/v) of the total membrane layer formulation, such as from 0.5% to 10% (v/v), including from 1% to 5% (v/v), for instance from 1.5% to 3% (v/v), and the like.

In certain embodiments, systems that include a sensor that includes an anti-inflammatory agent further include an inflammation detector. The inflammation detector may be configured to detect the presence or absence of inflammation. For example, the inflammation detector may be configured to detect the presence or absence of inflammation in the area surrounding the site of sensor insertion in a subject. In some instances, the inflammation detector is configured to detect the presence or absence of factors associated with inflammation as an indication of the presence or absence or inflammation. For instance, the inflammation detector is configured to detect the presence or absence of interleukin 1 as an indication of the presence or absence of inflammation. In certain cases, the system is configured to provide an indication of the presence or absence of inflammation to the subject. Upon detecting of inflammation, the system may be configured to provide an indication of the presence of inflammation to the subject. In some instances, if inflammation is not detected by the inflammation detector, the system provides an indication to the subject that inflammation is not present, or provides no indication of inflammation to the subject. As described above, in certain embodiments, inflammation may occur following sensor insertion in a subject, which may result in the Early Signal Attenuation (ESA) effect. Thus, in some instances, the system is configured to not display an analyte level on a display if the system detects inflammation in the tissues surrounding the sensor insertion site.

### Systems and Methods Using Clot Activators

Additional embodiments of the present disclosure relate to methods and devices for improving the signal response and/or stability of a sensor by inclusion of a clot activator disposed proximate to a working electrode of the sensor, such as in vivo analyte sensor, including, for example, continuous and/or automatic in vivo analyte sensors. For example, embodiments of the present disclosure provide for inclusion of a clot activator, resulting in an increase in the stability of the signal from the sensor over time and/or an increase in signal response. In certain embodiments, inclusion of the clot activator results in an increase in the stability of the signal from the sensor and/or an increase in signal response following insertion of in vivo biosensor in a user. Also provided are systems and methods of using the analyte sensors in analyte monitoring.

Embodiments of the present disclosure are based on the discovery that the addition of a clot activator to in vivo biosensors improves signal response and stability of the sensor. Biocompatible layers of embodiments of the present disclosure can include a clot activator, e.g., a compound or composition that increases the rate and/or amount of blood clotting in the tissues surrounding the site of the in vivo biosensor insertion in the user. In some cases, the clot activator is included in a membrane formulation disposed over the sensor. During use, the clot activator may diffuse out of the membrane formulation into the surrounding tissues. In some instances, the clot activator is applied to an exterior surface of the sensor, such that the clot activator is readily available on the surface of the sensor following insertion of the sensor in the user.

During in vivo use of the subject analyte sensors, a portion of the analyte sensor is inserted beneath a skin surface of a user. Following insertion of the analyte sensor, there may be a transient reduction in signal from the sensor. This results in variable data quality before the signal from the sensor stabilizes, resulting in a so-called Early Signal Attenuation (ESA) effect. Without being limited to any particular theory, in some cases, the ESA effect may be caused by the presence of blood clots in the area surrounding the site of sensor insertion. For instance, an increased presence of blood clots in the area surrounding the site of sensor insertion may lead to an increase in the local consumption of glucose in that area, resulting in a microenvironment with a reduced glucose level. The variability in the glucose level in the area surrounding the site of sensor insertion may cause a transient reduction in signal from the sensor (e.g., the so-called ESA effect).

Embodiments of the present disclosure provide for increased signal response and/or stability by decreasing the ESA effect. The result may be a reduction, and in some cases, complete elimination of the ESA effect. As such, embodiments that include the clot activator may provide for increased signal response and/or stability, such that substantially no ESA occurs following subcutaneous insertion of the analyte sensor. For example, as compared to sensors that do not include a clot activator, sensors that include a clot activator may have a reduction in the ESA effect for 30 min or more following subcutaneous insertion of the analyte sensor, such as 1 hour or more, including 2 hours or more, or 4 hours or more, or 6 hours or more, or 8 hours or more, or 10 hours or more, or 12 hours or more, for instance 14 hours or more, or 18 hours or more, or 24 hours or more, including 2 days or more, or 3 days or more, or 4 days or more, or 5 days or more, or 6 days or more, or 7 days or more, or 10 days or more, or 14 days or more following subcutaneous insertion of the analyte sensor. As such, sensors that include a clot activator may allow stable signal to be detected within a certain time period following subcutaneous insertion of the analyte sensor, such as 24 hours or less, or 18 hours or less, or 12 hours or less, or 8 hours or less, or 6 hours or less, or 5 hours or less, or 4 hours or less, or 3 hours or less, or 2 hours or less, or 1 hour or less, including 45 minutes or less, such as 30 minutes or less, for example, 15 minutes or less, or 10 minutes or less, or 5 minutes or less, or 3minutes or less, or 2 minutes or less, or 1 minute or less following subcutaneous insertion of the analyte sensor. In some instances, sensors that include a clot activator may allow stable signal to be detected immediately following subcutaneous insertion of the analyte sensor.

In certain embodiments of the present disclosure, inclusion of the clot activator results in an increase in the accuracy of the analyte measurements from the sensor. For example, inclusion of the clot activator may result in better correlation between the analyte concentration as determined by the in vivo analyte monitoring device (e.g., based on signals detected from the analyte sensor) and a reference analyte concentration. In certain instances, inclusion of the clot activator results in analyte concentrations as determined by the signals detected from the analyte sensor that are within 50% of a reference value, such as within 40% of the reference value, including within 30% of the reference value, or within 20% of the reference value, or within 10% of the reference value, or within 5% of the reference value, or within 2% of the reference value, or within 1% of the reference value. In some cases, the analyte sensor maintains its accuracy (e.g., is within a threshold percentage of a reference value, as described above) for 75% or more of the time during use, such as 80% or more, or 90% or more, including 95% or more, or 97% or more, or 99% or more of the time during use. As an alternative measure of accuracy, in some cases, inclusion of the clot activator results in analyte concentrations as determined by the signals detected from the analyte sensor that are within Zone A of the Clarke Error Grid Analysis. For example, inclusion of the clot activator may result in analyte concentrations as determined by the signals detected from the analyte sensor that are within Zone A of the Clarke Error Grid Analysis for 75% or more of the time during use, such as 80% or more, or 90% or more, including 95% or more, or 97% or more, or 99% or more of the time during use. In certain instances, inclusion of the clot activator results in analyte concentrations as determined by the signals detected from the analyte sensor that are within Zone A or Zone B of the Clarke Error Grid Analysis. For example, inclusion of the clot activator may result in analyte concentrations as determined by the signals detected from the analyte sensor that are within Zone A or Zone B of the Clarke Error Grid Analysis for 75% or more of the time during use, such as 80% or more, or 90% or more, including 95% or more, or 97% or more, or 99% or more of the time during use. Further information regarding the Clarke Error Grid Analysis is found in Clarke, W. L. et al. “Evaluating Clinical Accuracy of Systems for Self-Monitoring of Blood Glucose” Diabetes Care, vol. 10, no. 5, 1987:622-628.

In certain embodiments, sensors that include a clot activator have a sensitivity of 0.1 nA/mM or more, or 0.5 nA/mM or more, such as 1 nA/mM or more, including 1.5 nA/mM or more, for instance 2 nA/mM or more, or 2.5 nA/mM or more, or 3 nA/mM or more, or 3.5 nA/mM or more, or 4 nA/mM or more, or 4.5 nA/mM or more, or 5 nA/mM or more. In some cases, sensors that include a clot activator have a sensitivity ranging from 0.1 nA/mM to 5 nA/mM, such as from 0.1 nA/mM to 4.5 nA/mM, including from 0.1 nA/mM to 4 nA/mM, or from 0.2 nA/mM to 3.5 nA/mM, or from 0.2 nA/mM to 3 nA/mM, or from 0.3 nA/mM to 2.5 nA/mM, or from 0.3 nA/mM to 2 nA/mM.

In some cases, a sensor that includes a clot activator as disclosed herein has an initial sensitivity. The sensor may have a sensitivity that is 90% or more of the initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. For example, the sensor may maintain 95% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In some cases, the sensor maintains 97% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In certain instances, the sensor may maintain 99% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more.

Sensors that include a clot activator may also have increased linearity in signal over a wide range of analyte concentrations as compared to sensors that do not include a clot activator. In certain embodiments, sensors that include a clot activator have a substantially linear signal over a range of analyte concentrations. For example, sensors that include a clot activator may have a substantially linear signal over a range of blood glucose concentrations, such as from 10 to 1000 mg/dL, including from 25 to 700 mg/dL, for instance from 50 to 500 mg/dL, or from 50 to 300 mg/dL.

Examples of clot activators suitable for use with the subject devices, methods and kits include high surface area particles, such as, but not limited to, silica, diatomaceous earth (e.g., Celite), glass particles (e.g., powdered or micronized glass particles), kaolin, zeolites, combinations thereof, and the like. In some cases, clot activators may include procoagulants, such as, but not limited to, thrombin, fibrin, Prothrombin Complex Concentrate (PCC), recombinant human factor VIIa, combinations thereof, and the like.

The clot activator may be included in any component of a sensor that is inserted into a user during use or near the point of insertion. Embodiments include, but are not limited to, sensors that include a sensing layer having a clot activator, sensors that include a membrane layer having a clot activator, and sensors that include a clot activator disposed on an exterior surface of the sensor. In addition, the component formulation of a sensor (e.g., the sensing layer and/or membrane layer and/or clot activator formulation) may be contacted to the sensor in any of a variety of suitable ways, for example, but not limited to, dip coating, spray coating, drop deposition, and the like.

Additional embodiments of a sensor that may be suitably formulated with a clot activator are described in U.S. Pat. Nos. 5,262,035, 5,262,305, 6, 134,461, 6,143,164, 6,175,752, 6,338,790, 6,579,690, 6,605,200, 6,605,201, 6,654,625, 6,736,957, 6,746,582, 6,932,894, 7,090,756 as well as those described in U.S. patents application Ser. Nos. 11/701,138, 11/948,915, 12/625,185, 12/625,208, and 12/624,767, the disclosures of all of which are incorporated herein by reference in their entirety. Moreover, embodiments of the present disclosure may be incorporated into battery-powered or self-powered analyte sensors, in one embodiment the analyte sensor is a self-powered sensor, such as disclosed in U.S. patent application Ser. No. 12/393,921 (Publication No. 2010/0213057).

In some embodiments, the clot activator is formulated with a sensing layer that is disposed on a working electrode. The sensing layer may be described as the active chemical area of the biosensor. The sensing layer formulation, which can include a glucose-transducing agent, may include, for example, among other constituents, a redox mediator, such as, for example, a hydrogen peroxide or a transition metal complex, such as a ruthenium-containing complex or an osmium-containing complex, and an analyte-responsive enzyme, such as, for example, a glucose-responsive enzyme (e.g., glucose oxidase, glucose dehydrogenase, etc.) or lactate-responsive enzyme (e.g., lactate oxidase). In certain embodiments, the sensing layer includes glucose oxidase. The sensing layer may also include other optional components, such as, for example, a polymer and a bi-functional, short-chain, epoxide cross-linker, such as polyethylene glycol (PEG).

In certain instances, the analyte-responsive enzyme is distributed throughout the sensing layer. For example, the analyte-responsive enzyme may be distributed uniformly throughout the sensing layer, such that the concentration of the analyte-responsive enzyme is substantially the same throughout the sensing layer. In some cases, the sensing layer may have a homogeneous distribution of the analyte-responsive enzyme. In certain embodiments, the redox mediator is distributed throughout the sensing layer. For example, the redox mediator may be distributed uniformly throughout the sensing layer, such that the concentration of the redox mediator is substantially the same throughout the sensing layer. In some cases, the sensing layer may have a homogeneous distribution of the redox mediator. In certain embodiments, both the analyte-responsive enzyme and the redox mediator are distributed uniformly throughout the sensing layer, as described above.

Any suitable amount of clot activator may be used with the sensor, where the specifics will depend on, e.g., the particular sensing layer formulation, the particular membrane formulation, the type of clot activator, the site of sensor insertion, etc. In certain embodiments, the amount of the clot activator may range from 0.1 μg to 100 mg, such as from 1 μg to 10 mg, including from 10 μg to 1000 μg, for instance from 50 μg to 500 μg, and the like. In certain cases, only the membrane layer includes the clot activator. For instance, the clot activator may only be included in the membrane layer and substantially excluded from any of the other layers of the sensor, such as, but not limited to, the sensing layer. In certain embodiments, the clot activator is applied to the exterior surface of the sensor, such as by dip coating, spray coating, drop deposition, and the like. In these embodiments, the clot activator formulation may include the clot activator in a amount ranging from 0.1 μg to 100 mg, such as from 1 μg to 10 mg, including from 10 μg to 1000 μg, for instance from 50 μg to 500 μg, and the like.

### Systems and Methods Using Immunosuppressants

Additional embodiments of the present disclosure relate to methods and devices for improving the lifespan of a sensor by inclusion of an immunosuppressant, where the sensor is an in vivo analyte sensor, including, for example, continuous and/or automatic in vivo analyte sensors. For example, embodiments of the present disclosure provide for inclusion of an immunosuppressant, resulting in an increase in the stability of the signal from the sensor over time and/or an increase in signal response. In some instances, inclusion of the immunosuppressant results in an increase in the stability of the signal from the sensor over time, thus increasing the lifespan of the sensor. Also provided are systems and methods of using the analyte sensors in analyte monitoring.

Embodiments of the present disclosure are based on the discovery that the addition of an immunosuppressant to in vivo biosensors improves the stability of the sensor. Biocompatible layers of embodiments of the present disclosure can include immunosuppressant, e.g., compounds or compositions that decrease occurrence and/or severity of the body's immune response to foreign objects in the body, such as an in vivo biosensor inserted in a user. In some cases, the immunosuppressant is included in a membrane formulation disposed over the sensor. During use, the immunosuppressant may diffuse out of the membrane formulation into the surrounding tissues. In some instances, the immunosuppressant is applied to an exterior surface of the sensor, such that the immunosuppressant is readily available on the surface of the sensor following insertion of the sensor in the user.

During in vivo use of the subject analyte sensors, a portion of the analyte sensor is inserted beneath a skin surface of a user. Following insertion of the analyte sensor, the sensor may produce a stable detectable signal for a certain period of time, such as 1 day or more, 3 days or more, or 5 days or more. After a certain period of time, the sensor may need to be replaced with a new sensor. Without being limited to any particular theory, for example, the user may experience an immune response to the sensor, such as, redness, pain, tenderness, or swelling at the sensor insertion site. In some instances, foreign-body giant cells and/or other immune system tissues may build up around the sensor insertion site, resulting in a decreased diffusion of blood or interstitial fluid across the sensor membrane. This in turn may result in decreased sensor signal and/or decreased sensor sensitivity, which in some instances may lead to inaccurate sensor measurements.

Embodiments of the present disclosure provide for increased signal response and/or stability. The result may be an increase in the lifespan of the sensor. For example, embodiments of the present disclosure include sensors that produce stable detectable signals for a longer period of time during use. As such, embodiments that include the immunosuppressant may provide for increased signal response and/or stability, such that the analyte sensor has an increased lifespan. For example, as compared to sensors that do not include an immunosuppressant, sensors that include an immunosuppressant may have a lifespan of 1 day or more, including 2 days or more, or 3 days or more, or 4 days or more, or 5 days or more, or 6 days or more, or 7 days or more, or 10 days or more, or 14 days or more following subcutaneous insertion of the analyte sensor. As such, sensors that include an immunosuppressant may allow stable signal to be detected for a certain time period following subcutaneous insertion of the analyte sensor, such as 1 day or more, including 2 days or more, or 3 days or more, or 4 days or more, or 5 days or more, or 6 days or more, or 7 days or more, or 10 days or more, or 14 days or more following subcutaneous insertion of the analyte sensor.

In certain embodiments of the present disclosure, inclusion of the immunosuppressant results in an increase in the accuracy of the analyte measurements from the sensor for the lifespan of the sensor. For example, inclusion of the immunosuppressant may result in better correlation between the analyte concentration as determined by the in vivo analyte monitoring device (e.g., based on signals detected from the analyte sensor) and a reference analyte concentration. In certain instances, inclusion of the immunosuppressant results in analyte concentrations as determined by the signals detected from the analyte sensor that are within 50% of a reference value, such as within 40% of the reference value, including within 30% of the reference value, or within 20% of the reference value, or within 10% of the reference value, or within 5% of the reference value, or within 2% of the reference value, or within 1% of the reference value. In some cases, the analyte sensors maintains its accuracy (e.g., is within a threshold percentage of a reference value, as described above) for 75% or more of the time during use, such as 80% or more, or 90% or more, including 95% or more, or 97% or more, or 99% or more of the time during use. As an alternative measure of accuracy, in some cases, inclusion of the immunosuppressant results in analyte concentrations as determined by the signals detected from the analyte sensor that are within Zone A of the Clarke Error Grid Analysis. For example, inclusion of the immunosuppressant may result in analyte concentrations as determined by the signals detected from the analyte sensor that are within Zone A of the Clarke Error Grid Analysis for 75% or more of the time during use, such as 80% or more, or 90% or more, including 95% or more, or 97% or more, or 99% or more of the time during use. In certain instances, inclusion of the immunosuppressant results in analyte concentrations as determined by the signals detected from the analyte sensor that are within Zone A or Zone B of the Clarke Error Grid Analysis. For example, inclusion of the immunosuppressant may result in analyte concentrations as determined by the signals detected from the analyte sensor that are within Zone A or Zone B of the Clarke Error Grid Analysis for 75% or more of the time during use, such as 80% or more, or 90% or more, including 95% or more, or 97% or more, or 99% or more of the time during use. Further information regarding the Clarke Error Grid Analysis is found in Clarke, W. L. et al. “Evaluating Clinical Accuracy of Systems for Self-Monitoring of Blood Glucose” Diabetes Care, vol. 10, no. 5, 1987:622-628.

In certain embodiments, sensors that include an immunosuppressant have a sensitivity of 0.1 nA/mM or more, or 0.5 nA/mM or more, such as 1 nA/mM or more, including 1.5 nA/mM or more, for instance 2 nA/mM or more, or 2.5 nA/mM or more, or 3 nA/mM or more, or 3.5 nA/mM or more, or 4 nA/mM or more, or 4.5 nA/mM or more, or 5 nA/mM or more. In some cases, sensors that include an immunosuppressant have a sensitivity ranging from 0.1 nA/mM to 5 nA/mM, such as from 0.1 nA/mM to 4.5 nA/mM, including from 0.1 nA/mM to 4 nA/mM, or from 0.2 nA/mM to 3.5 nA/mM, or from 0.2 nA/mM to 3 nA/mM, or from 0.3 nA/mM to 2.5 nA/mM, or from 0.3 nA/mM to 2 nA/mM.

In some instances, inclusion of an immunosuppressant provides for increased signal response and/or stability over the life of the sensor. The result may be an increase in the lifespan of the sensor as compared to sensors that do not include an immunosuppressant. In some cases, a sensor that includes an immunosuppressant as disclosed herein has an initial sensitivity. The sensor may have a sensitivity that is 90% or more of the initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. For example, the sensor may maintain 95% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In some cases, the sensor maintains 97% or more of its initial sensitivity after 1 day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more. In certain instances, the sensor may maintain 99% or more of its initial sensitivity after 1day or more, such as 2 days or more, 3 days or more, 4 days or more, 5 days or more, 6 days or more, 7 days or more, 10 days or more, 14 days or more, 1 month or more, 2 months or more, 4 months or more, 6 months or more, 9 months or more, or 1 year or more.

Sensors that include an immunosuppressant may also have increased linearity in signal over a wide range of analyte concentrations as compared to sensors that do not include an immunosuppressant. In certain embodiments, sensors that include an immunosuppressant have a substantially linear signal over a range of analyte concentrations. For example, sensors that include an immunosuppressant may have a substantially linear signal over a range of blood glucose concentrations, such as from 10 to 1000 mg/dL, including from 25 to 700 mg/dL, for instance from 50 to 500 mg/dL, or from 50 to 300 mg/dL.

In certain embodiments, a sensor that includes an immunosuppressant as disclosed herein has an increased lifespan as compared to a sensor that does not include an immunosuppressant. For example, a sensor that includes an immunosuppressant may produce accurate, detectable signals for 1 day or more, such as 2 days or more, or 3 days or more, including 4 days or more, 5 days or more, 6 days or more, 7 days or more, or 10 days or more, or 14 days or more, or 3 weeks or more, or 1 month or more. Stated another way, a sensor that includes an immunosuppressant may be used by the user for 1 day or more, such as 2 days or more, or 3 days or more, including 4 days or more, 5 days or more, 6 days or more, 7 days or more, or 10 days or more, or 14 days or more, or 3 weeks or more, or 1 month or more before needing to be replaced with a new sensor.

Examples of immunosuppressants suitable for use with the subject devices, methods and kits include, but are not limited to, mammalian target of rapamycin (mTOR) inhibitors (e.g., everolimus, sirolimus, etc.), and the like. Other immunosuppressants suitable for use with the subject devices, methods and kits include, but are not limited to, glucocorticoids (e.g., hydrocortisone, prednisone, prednisolone, methylprednisolone, etc.), drugs acting on immunophilins (e.g., ciclosporin, tacrolimus, sirolimus, everolimus, etc.), other immunosuppressive drugs (e.g., interferons, such as IFN-β; tumor necrosis factor-alpha (TNF-α) binding proteins, such as infliximab (Remicade), etanercept (Enbrel), or adalimumab (Humira); etc.), combinations thereof, and the like.

The immunosuppressant may be included in any component of a sensor that is inserted into a user during use or near the point of insertion. Embodiments include, but are not limited to, sensors that include a sensing layer having an immunosuppressant, sensors that include a membrane layer having an immunosuppressant, sensors that include an immunosuppressant disposed on an exterior surface of the sensor, and sensors that include an exterior layer that includes an immunosuppressant. In addition, the component formulation of a sensor (e.g., the sensing layer and/or membrane layer and/or immunosuppressant formulation) may be contacted to the sensor in any of a variety of suitable ways, for example, but not limited to, dip coating, spray coating, drop deposition, and the like. In certain instances, the immunosuppressant is included in an exterior layer of the sensor. For example, the immunosuppressant may be included in a layer (e.g., a coating) disposed on an exterior surface of the sensor substrate. The layer that includes the immunosuppressant may be of any suitable formulation that is compatible with the immunosuppressant, the sensor and in vivo use of the sensor in a user's body. In some cases, the layer including the immunosuppressant is a polymer layer, such as, but not limited to, polyvinylidene fluoride (PVDF), hexafluoroproplylene (HFP), polyvinylidene fluoride-hexafluoroproplylene (PVDF-HFP), polyvinyl fluoride (PVF), polytetrafluoroethylene (PTFE), polychlorotrifluoroethylene (PCTFE), perfluoroalkoxy polymer (PFA), fluorinated ethylene-propylene (FEP), polyethylenetetrafluoroethylene (ETFE), polyethylenechlorotrifluoroethylene (ECTFE), Nafion, combinations thereof, and the like. For example, the layer including the immunosuppressant may include a layer of PVDF-HFP applied to an exterior surface of the sensor substrate. The immunosuppressant may be incorporated into the polymer layer and may elute out of the polymer layer during use, such that the immunosuppressant is delivered to the tissues surrounding the insertion site of the analyte sensor during use.

Additional embodiments of a sensor that may be suitably formulated with an immunosuppressant are described in U.S. Pat. Nos. 5,262,035, 5,262,305, 6,134,461, 6,143,164, 6,175,752, 6,338,790, 6,579,690, 6,605,200, 6,605,201, 6,654,625, 6,736,957, 6,746,582, 6,932,894, 7,090,756 as well as those described in U.S. patents application Ser. Nos. 11/701,138, 11/948,915, 12/625,185, 12/625,208, and 12/624,767, the disclosures of all of which are incorporated herein by reference in their entirety. Moreover, embodiments of the present disclosure may be incorporated into battery-powered or self-powered analyte sensors, in one embodiment the analyte sensor is a self-powered sensor, such as disclosed in U.S. patent application Ser. No. 12/393,921 (Publication No. 2010/0213057).

In some embodiments, the immunosuppressant is formulated with a sensing layer that is disposed on a working electrode. The sensing layer may be described as the active chemical area of the biosensor. The sensing layer formulation, which can include a glucose-transducing agent, may include, for example, among other constituents, a redox mediator, such as, for example, a hydrogen peroxide or a transition metal complex, such as a ruthenium-containing complex or an osmium-containing complex, and an analyte-responsive enzyme, such as, for example, a glucose-responsive enzyme (e.g., glucose oxidase, glucose dehydrogenase, etc.) or lactate-responsive enzyme (e.g., lactate oxidase). In certain embodiments, the sensing layer includes glucose oxidase. The sensing layer may also include other optional components, such as, for example, a polymer and a bi-functional, short-chain, epoxide cross-linker, such as polyethylene glycol (PEG).

In certain instances, the analyte-responsive enzyme is distributed throughout the sensing layer. For example, the analyte-responsive enzyme may be distributed uniformly throughout the sensing layer, such that the concentration of the analyte-responsive enzyme is substantially the same throughout the sensing layer. In some cases, the sensing layer may have a homogeneous distribution of the analyte-responsive enzyme. In certain embodiments, the redox mediator is distributed throughout the sensing layer. For example, the redox mediator may be distributed uniformly throughout the sensing layer, such that the concentration of the redox mediator is substantially the same throughout the sensing layer. In some cases, the sensing layer may have a homogeneous distribution of the redox mediator. In certain embodiments, both the analyte-responsive enzyme and the redox mediator are distributed uniformly throughout the sensing layer, as described above.

Any suitable proportion of immunosuppressant may be used with the sensor, where the specifics will depend on, e.g., the particular sensing layer formulation, the particular membrane formulation, the composition of the polymer layer that includes the immunosuppressant, the site of sensor insertion, etc. In certain embodiments, the concentration of the immunosuppressant may range from 0.1% to 25% (v/v) of the immunosuppressant layer formulation, such as from 0.5% to 10% (v/v), including from 1% to 5% (v/v), for instance from 1.5% to 3% (v/v), and the like. In certain cases, only the membrane layer includes the immunosuppressant. For instance, the immunosuppressant may only be included in the membrane layer and substantially excluded from any of the other layers of the sensor, such as, but not limited to, the sensing layer. In certain embodiments, the immunosuppressant is applied to the exterior surface of the sensor, such as by dip coating, spray coating, drop deposition, and the like. In these embodiments, the immunosuppressant formulation may include the immunosuppressant in a concentration ranging from 0.1% to 25% (v/v) of the total membrane layer formulation, such as from 0.5% to 10% (v/v), including from 1% to 5% (v/v), for instance from 1.5% to 3% (v/v), and the like. In other embodiments, as described above, the immunosuppressant is included in a polymer layer disposed on an exterior surface of the sensor substrate.

### Electrochemical Sensors

Embodiments of the present disclosure relate to methods and devices for detecting at least one analyte, including glucose, in body fluid. Embodiments relate to the continuous and/or automatic in vivo monitoring of the level of one or more analytes using a continuous analyte monitoring system that includes an analyte sensor at least a portion of which is to be positioned beneath a skin surface of a user for a period of time and/or the discrete monitoring of one or more analytes using an in vitro blood glucose (“BG”) meter and an analyte test strip. Embodiments include combined or combinable devices, systems and methods and/or transferring data between an in vivo continuous system and an in vivo system. In some embodiments, the systems, or at least a portion of the systems, are integrated into a single unit.

A sensor as described herein may be an in vivo sensor or an in vitro sensor (i.e., a discrete monitoring test strip). Such a sensor can be formed on a substrate, e.g., a substantially planar substrate. In certain embodiments, the sensor is a wire, e.g., a working electrode wire inner portion with one or more other electrodes associated (e.g., on, including wrapped around) therewith. The sensor may also include at least one counter electrode (or counter/reference electrode) and/or at least one reference electrode or at least one reference/counter electrode.

Accordingly, embodiments include analyte monitoring devices and systems that include an analyte sensor at least a portion of which is positionable beneath the skin surface of the user for the in vivo detection of an analyte, including glucose, lactate, and the like, in a body fluid. Embodiments include wholly implantable analyte sensors and analyte sensors in which only a portion of the sensor is positioned under the skin and a portion of the sensor resides above the skin, e.g., for contact to a sensor control unit (which may include a transmitter), a receiver/display unit, transceiver, processor, etc. The sensor may be, for example, subcutaneously positionable in a user for the continuous or periodic monitoring of a level of an analyte in the user's interstitial fluid. For the purposes of this description, continuous monitoring and periodic monitoring will be used interchangeably, unless noted otherwise. The sensor response may be correlated and/or converted to analyte levels in blood or other fluids. In certain embodiments, an analyte sensor may be positioned in contact with interstitial fluid to detect the level of glucose, which detected glucose may be used to infer the glucose level in the user's bloodstream. Analyte sensors may be insertable into a vein, artery, or other portion of the body containing fluid. Embodiments of the analyte sensors may be configured for monitoring the level of the analyte over a time period which may range from seconds, minutes, hours, days, weeks, to months, or longer.

In certain embodiments, the analyte sensors, such as glucose sensors, are capable of in vivo detection of an analyte for one hour or more, e.g., a few hours or more, e.g., a few days or more, e.g., three or more days, e.g., five days or more, e.g., seven days or more, e.g., several weeks or more, or one month or more. Future analyte levels may be predicted based on information obtained, e.g., the current analyte level at time to, the rate of change of the analyte, etc. Predictive alarms may notify the user of a predicted analyte levels that may be of concern in advance of the user's analyte level reaching the future predicted analyte level. This provides the user an opportunity to take corrective action.

In an electrochemical embodiment, the sensor is placed, transcutaneously, for example, into a subcutaneous site such that subcutaneous fluid of the site comes into contact with the sensor. In other in vivo embodiments, placement of at least a portion of the sensor may be in a blood vessel. The sensor operates to electrolyze an analyte of interest in the subcutaneous fluid or blood such that a current is generated between the working electrode and the counter electrode. A value for the current associated with the working electrode is determined. If multiple working electrodes are used, current values from each of the working electrodes may be determined. A microprocessor may be used to collect these periodically determined current values or to further process these values.

If an analyte concentration is successfully determined, it may be displayed, stored, transmitted, and/or otherwise processed to provide useful information. By way of example, raw signal or analyte concentrations may be used as a basis for determining a rate of change in analyte concentration, which should not change at a rate greater than a predetermined threshold amount. If the rate of change of analyte concentration exceeds the predefined threshold, an indication maybe displayed or otherwise transmitted to indicate this fact. In certain embodiments, an alarm is activated to alert a user if the rate of change of analyte concentration exceeds the predefined threshold.

As demonstrated herein, the methods of the present disclosure are useful in connection with a device that is used to measure or monitor an analyte (e.g., glucose), such as any such device described herein. These methods may also be used in connection with a device that is used to measure or monitor another analyte (e.g., ketones, ketone bodies, HbA1c, and the like), including oxygen, carbon dioxide, proteins, drugs, or another moiety of interest, for example, or any combination thereof, found in bodily fluid, including subcutaneous fluid, dermal fluid (sweat, tears, and the like), interstitial fluid, or other bodily fluid of interest, for example, or any combination thereof. In general, the device is in good contact, such as thorough and substantially continuous contact, with the bodily fluid.

According to embodiments of the present disclosure, the measurement sensor is one suited for electrochemical measurement of analyte concentration, for example glucose concentration, in a bodily fluid. In these embodiments, the measurement sensor includes at least a working electrode and a counter electrode. Other embodiments may further include a reference electrode. The working electrode is typically associated with a glucose-responsive enzyme. A mediator may also be included. In certain embodiments, hydrogen peroxide, which may be characterized as a mediator, is produced by a reaction of the sensor and may be used to infer the concentration of glucose. In some embodiments, a mediator is added to the sensor by a manufacturer, i.e., is included with the sensor prior to use. The redox mediator may be disposed relative to the working electrode and is capable of transferring electrons between a compound and a working electrode, either directly or indirectly. The redox mediator may be, for example, immobilized on the working electrode, e.g., entrapped on a surface or chemically bound to a surface.

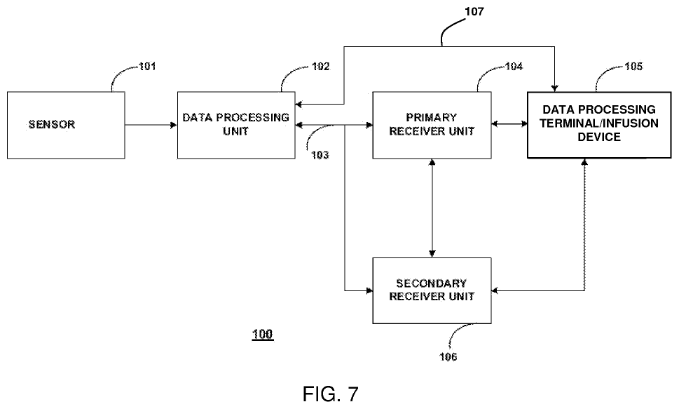

FIG. 7 shows a data monitoring and management system such as, for example, an analyte (e.g., glucose) monitoring system 100 in accordance with certain embodiments. Aspects of the subject disclosure are further described primarily with respect to glucose monitoring devices and systems, and methods of glucose detection, for convenience only and such description is in no way intended to limit the scope of the embodiments. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

The analyte monitoring system 100 includes an analyte sensor 101, a data processing unit 102 connectable to the sensor 101, and a primary receiver unit 104. In some instances, the primary receiver unit 104 is configured to communicate with the data processing unit 102 via a communication link 103. In certain embodiments, the primary receiver unit 104 may be further configured to transmit data to a data processing terminal 105 to evaluate or otherwise process or format data received by the primary receiver unit 104. The data processing terminal 105 may be configured to receive data directly from the data processing unit 102 via a communication link 107, which may optionally be configured for bi-directional communication. Further, the data processing unit 102 may include a transmitter or a transceiver to transmit and/or receive data to and/or from the primary receiver unit 104 and/or the data processing terminal 105 and/or optionally a secondary receiver unit 106.

Also shown in FIG. 7 is an optional secondary receiver unit 106 which is operatively coupled to the communication link 103 and configured to receive data transmitted from the data processing unit 102. The secondary receiver unit 106 may be configured to communicate with the primary receiver unit 104, as well as the data processing terminal 105. In certain embodiments, the secondary receiver unit 106 may be configured for bi-directional wireless communication with each of the primary receiver unit 104 and the data processing terminal 105. As discussed in further detail below, in some instances, the secondary receiver unit 106 may be a de-featured receiver as compared to the primary receiver unit 104, for instance, the secondary receiver unit 106 may include a limited or minimal number of functions and features as compared with the primary receiver unit 104. As such, the secondary receiver unit 106 may include a smaller (in one or more, including all, dimensions), compact housing or embodied in a device including a wrist watch, arm band, PDA, mp3 player, cell phone, etc., for example. Alternatively, the secondary receiver unit 106 may be configured with the same or substantially similar functions and features as the primary receiver unit 104. The secondary receiver unit 106 may include a docking portion configured to mate with a docking cradle unit for placement by, e.g., the bedside for night time monitoring, and/or a bi-directional communication device. A docking cradle may recharge a power supply.

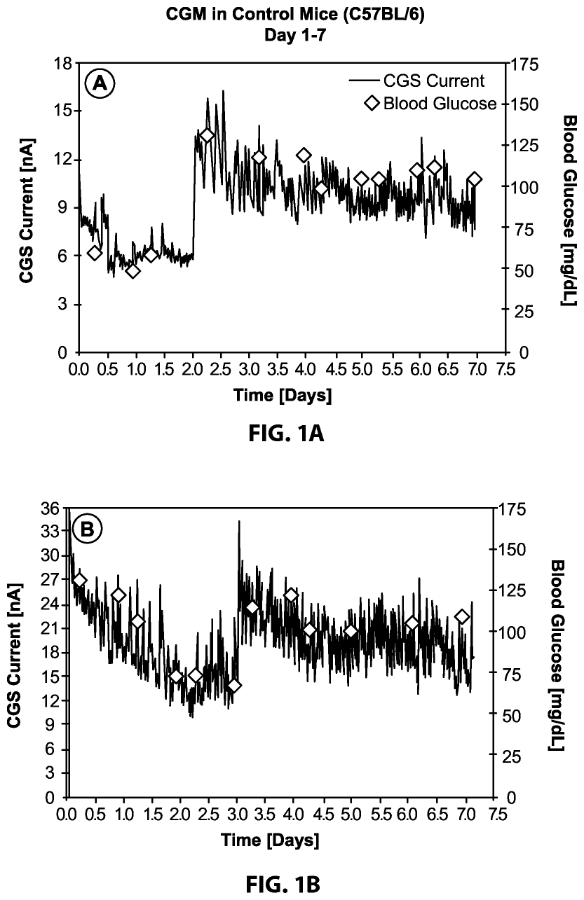

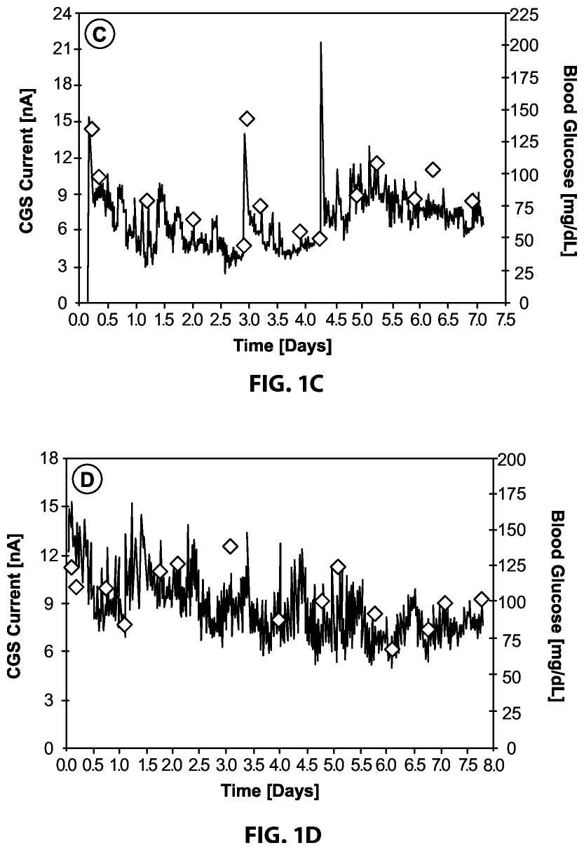

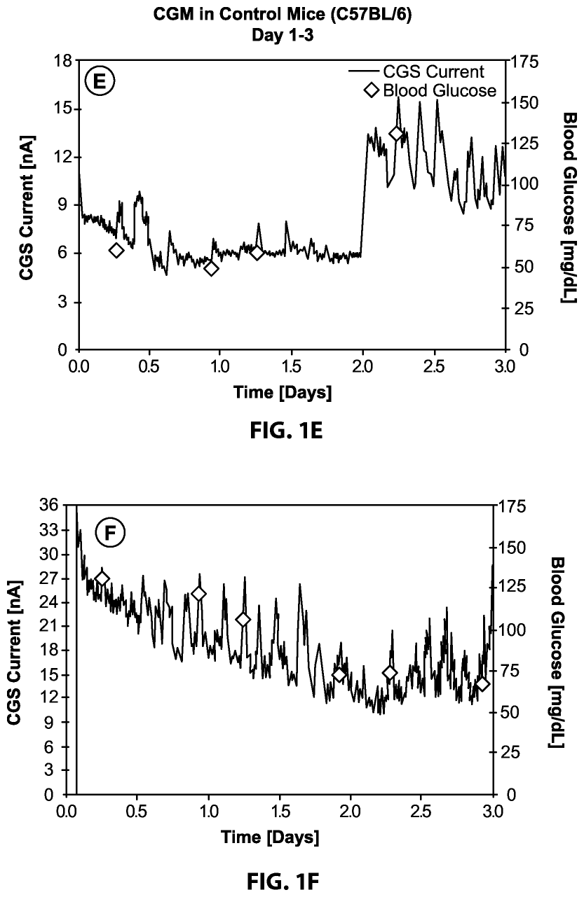

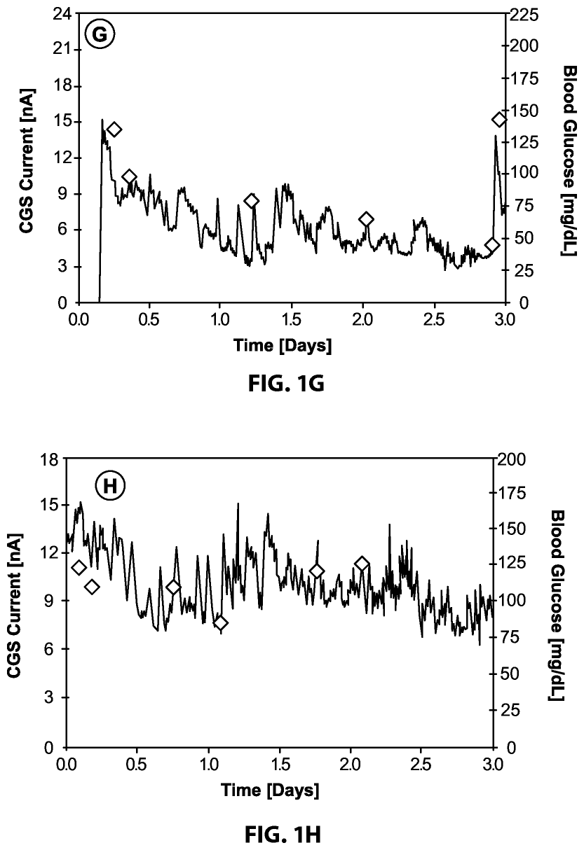

Only one analyte sensor 101, data processing unit 102 and data processing terminal 105 are shown in the embodiment of the analyte monitoring system 100 illustrated in FIGS. 1A-H. However, it will be appreciated by one of ordinary skill in the art that the analyte monitoring system 100 may include more than one sensor 101 and/or more than one data processing unit 102, and/or more than one data processing terminal 105. Multiple sensors may be positioned in a user for analyte monitoring at the same or different times. In certain embodiments, analyte information obtained by a first sensor positioned in a user may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 100 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 100. For example, unique IDs, communication channels, and the like, may be used.

In certain embodiments, the sensor 101 is physically positioned in or on the body of a user whose analyte level is being monitored. The sensor 101 may be configured to at least periodically sample the analyte level of the user and convert the sampled analyte level into a corresponding signal for transmission by the data processing unit 102. The data processing unit 102 is coupleable to the sensor 101 so that both devices are positioned in or on the user's body, with at least a portion of the analyte sensor 101 positioned transcutaneously. The data processing unit may include a fixation element, such as an adhesive or the like, to secure it to the user's body. A mount (not shown) attachable to the user and mateable with the data processing unit 102 may be used. For example, a mount may include an adhesive surface. The data processing unit 102 performs data processing functions, where such functions may include, but are not limited to, filtering and encoding of data signals, each of which corresponds to a sampled analyte level of the user, for transmission to the primary receiver unit 104 via the communication link 103. In some embodiments, the sensor 101 or the data processing unit 102 or a combined sensor/data processing unit may be wholly implantable under the skin surface of the user.

In certain embodiments, the primary receiver unit 104 may include an analog interface section including an RF receiver and an antenna that is configured to communicate with the data processing unit 102 via the communication link 103, and a data processing section for processing the received data from the data processing unit 102 including data decoding, error detection and correction, data clock generation, data bit recovery, etc., or any combination thereof.

In operation, the primary receiver unit 104 in certain embodiments is configured to synchronize with the data processing unit 102 to uniquely identify the data processing unit 102, based on, for example, an identification information of the data processing unit 102, and thereafter, to periodically receive signals transmitted from the data processing unit 102 associated with the monitored analyte levels detected by the sensor 101.

Referring again to FIG. 7, the data processing terminal 105 may include a personal computer, a portable computer including a laptop or a handheld device (e.g., a personal digital assistant (PDA), a telephone including a cellular phone (e.g., a multimedia and Internet-enabled mobile phone including an iPhone™, a Blackberry®, an Android™ phone, or similar phone), an mp3 player (e.g., an iPOD™, etc.), a pager, and the like), and/or a drug delivery device (e.g., an infusion device), each of which may be configured for data communication with the receiver via a wired or a wireless connection. Additionally, the data processing terminal 105 may further be connected to a data network (not shown) for storing, retrieving, updating, and/or analyzing data corresponding to the detected analyte level of the user.

The data processing terminal 105 may include a drug delivery device (e.g., an infusion device) such as an insulin infusion pump or the like, which may be configured to administer a drug (e.g., insulin) to the user, and which may be configured to communicate with the primary receiver unit 104 for receiving, among others, the measured analyte level. Alternatively, the primary receiver unit 104 may be configured to integrate an infusion device therein so that the primary receiver unit 104 is configured to administer an appropriate drug (e.g., insulin) to users, for example, for administering and modifying basal profiles, as well as for determining appropriate boluses for administration based on, among others, the detected analyte levels received from the data processing unit 102. An infusion device may be an external device or an internal device, such as a device wholly implantable in a user.

In certain embodiments, the data processing terminal 105, which may include an infusion device, e.g., an insulin pump, may be configured to receive the analyte signals from the data processing unit 102, and thus, incorporate the functions of the primary receiver unit 104 including data processing for managing the user's insulin therapy and analyte monitoring. In certain embodiments, the communication link 103, as well as one or more of the other communication interfaces shown in FIG. 7, may use one or more wireless communication protocols, such as, but not limited to: an RF communication protocol, an infrared communication protocol, a Bluetooth enabled communication protocol, an 802.11x wireless communication protocol, or an equivalent wireless communication protocol which would allow secure, wireless communication of several units (for example, per Health Insurance Portability and Accountability Act (HIPPA) requirements), while avoiding potential data collision and interference.

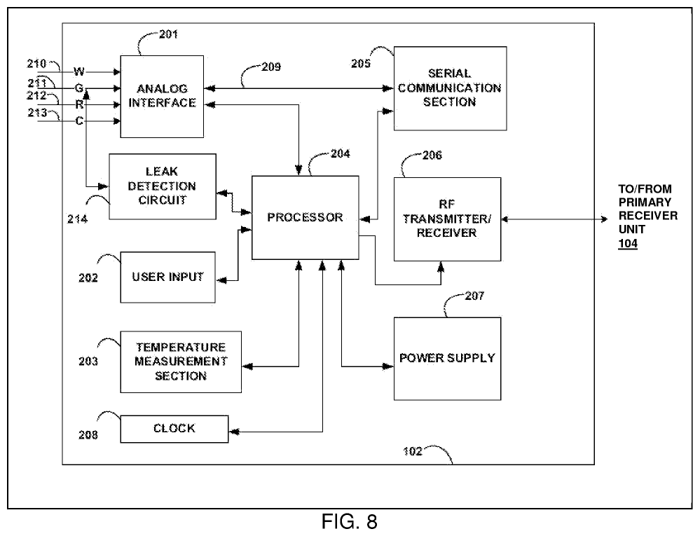

FIG. 8 shows a block diagram of an embodiment of a data processing unit 102 of the analyte monitoring system shown in FIG. 7. User input and/or interface components may be included or a data processing unit may be free of user input and/or interface components. In certain embodiments, one or more application-specific integrated circuits (ASIC) may be used to implement one or more functions or routines associated with the operations of the data processing unit (and/or receiver unit) using for example one or more state machines and buffers.

As can be seen in the embodiment of FIG. 8, the analyte sensor 101 (FIG. 7) includes four contacts, three of which are electrodes: a work electrode (W) 210, a reference electrode (R) 212, and a counter electrode (C) 213, each operatively coupled to the analog interface 201 of the data processing unit 102. This embodiment also shows an optional guard contact (G) 211. Fewer or greater electrodes may be employed. For example, the counter and reference electrode functions may be served by a single counter/reference electrode. In some cases, there may be more than one working electrode and/or reference electrode and/or counter electrode, etc.

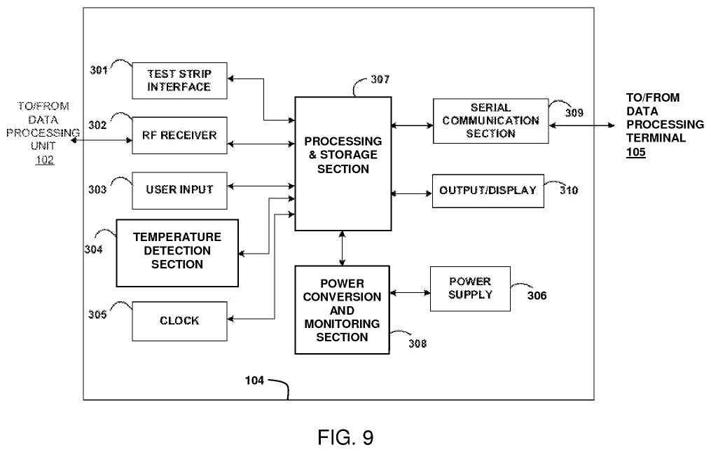

FIG. 9 is a block diagram of an embodiment of a receiver/monitor unit such as the primary receiver unit 104 of the analyte monitoring system shown in FIG. 7. The primary receiver unit 104 includes one or more of: a test strip interface 301, an RF receiver 302, a user input 303, an optional temperature detection section 304, and a clock 305, each of which is operatively coupled to a processing and storage section 307. The primary receiver unit 104 also includes a power supply 306 operatively coupled to a power conversion and monitoring section 308. Further, the power conversion and monitoring section 308 is also coupled to the processing and storage section 307. Moreover, also shown are a receiver serial communication section 309, and an output 310, each operatively coupled to the processing and storage section 307. The primary receiver unit 104 may include user input and/or interface components or may be free of user input and/or interface components.

In certain embodiments, the test strip interface 301 includes an analyte testing portion (e.g., a glucose level testing portion) to receive a blood (or other body fluid sample) analyte test or information related thereto. For example, the test strip interface 301 may include a test strip port to receive a test strip (e.g., a glucose test strip). The device may determine the analyte level of the test strip, and optionally display (or otherwise notice) the analyte level on the output 310 of the primary receiver unit 104. Any suitable test strip may be employed, e.g., test strips that only require a very small amount (e.g., 3 microliters or less, e.g., 1 microliter or less, e.g., 0.5 microliters or less, e.g., 0.1 microliters or less), of applied sample to the strip in order to obtain accurate glucose information. Embodiments of test strips include, e.g., FreeStyle® blood glucose test strips from Abbott Diabetes Care Inc. (Alameda, CA). Glucose information obtained by an in vitro glucose testing device may be used for a variety of purposes, computations, etc. For example, the information may be used to calibrate sensor 101, confirm results of sensor 101 to increase the confidence thereof (e.g., in instances in which information obtained by sensor 101 is employed in therapy related decisions), etc.

In further embodiments, the data processing unit 102 and/or the primary receiver unit 104 and/or the secondary receiver unit 106, and/or the data processing terminal/infusion device 105 may be configured to receive the analyte value wirelessly over a communication link from, for example, a blood glucose meter. In further embodiments, a user manipulating or using the analyte monitoring system 100 (FIG. 7) may manually input the analyte value using, for example, a user interface (for example, a keyboard, keypad, voice commands, and the like) incorporated in one or more of the data processing unit 102, the primary receiver unit 104, secondary receiver unit 106, or the data processing terminal/infusion device 105.

Additional detailed descriptions are provided in U.S. Pat. Nos. 5,262,035; 5,264,104; 5,262,305; 5,320,715; 5,593,852; 6,175,752; 6,650,471; 6,746, 582, and 7,811,231, each of which is incorporated herein by reference in their entirety.

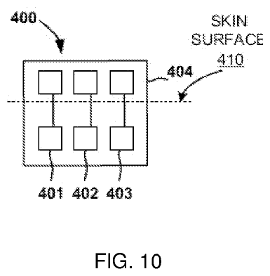

FIG. 10 schematically shows an embodiment of an analyte sensor 400 in accordance with the embodiments of the present disclosure. This sensor embodiment includes electrodes 401, 402 and 403 on a base 404. Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like. Materials include, but are not limited to, any one or more of aluminum, carbon (including graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The analyte sensor 400 may be wholly implantable in a user or may be configured so that only a portion is positioned within (internal) a user and another portion outside (external) a user. For example, the sensor 400 may include a first portion positionable above a surface of the skin 410, and a second portion positioned below the surface of the skin. In such embodiments, the external portion may include contacts (connected to respective electrodes of the second portion by traces) to connect to another device also external to the user such as a transmitter unit. While the embodiment of FIG. 10 shows three electrodes side-by-side on the same surface of base 404, other configurations are contemplated, e.g., fewer or greater electrodes, some or all electrodes on different surfaces of the base or present on another base, some or all electrodes stacked together, electrodes of differing materials and dimensions, etc.

In certain embodiments, the analyte sensor has a first portion positionable above a surface of the skin, and a second portion that includes an insertion tip positionable below the surface of the skin, e.g., penetrating through the skin and into, e.g., the subcutaneous space, in contact with the user's biofluid, such as interstitial fluid. Contact portions of a working electrode, a reference electrode, and a counter electrode are positioned on the first portion of the sensor situated above the skin surface. A working electrode, a reference electrode, and a counter electrode may be present on the second portion of the sensor, such as at the insertion tip. Traces may be provided from the electrodes at the tip to the contacts. It is to be understood that greater or fewer electrodes may be provided on a sensor. For example, a sensor may include more than one working electrode and/or the counter and reference electrodes may be a single counter/reference electrode, etc.

In certain embodiments, the electrodes of the sensor as well as the substrate and the dielectric layers are provided in a layered configuration or construction. For example, in one embodiment, the sensor (such as the analyte sensor unit 101 of FIG. 7), includes a substrate layer, and a first conducting layer such as carbon, gold, etc., disposed on at least a portion of the substrate layer, and which may provide the working electrode. Also disposed on at least a portion of the first conducting layer may be a sensing layer.

A first insulation layer, such as a first dielectric layer in certain embodiments, may be disposed or layered on at least a portion of the first conducting layer, and further, a second conducting layer may be disposed or stacked on top of at least a portion of the first insulation layer (or dielectric layer). The second conducting layer may provide the reference electrode, and in one aspect, may include a layer of silver/silver chloride (Ag/AgCl), gold, etc.

A second insulation layer, such as a second dielectric layer in certain embodiments, may be disposed or layered on at least a portion of the second conducting layer. Further, a third conducting layer may be disposed on at least a portion of the second insulation layer and may provide the counter electrode. Finally, a third insulation layer may be disposed or layered on at least a portion of the third conducting layer. In this manner, the sensor may be layered such that at least a portion of each of the conducting layers is separated by a respective insulation layer (for example, a dielectric layer). In certain instances, some or all of the layers may have the same or different lengths and/or widths.

In certain embodiments, some or all of the electrodes may be provided on the same side of the substrate in the layered construction as described above, or alternatively, may be provided in a co-planar manner such that two or more electrodes may be positioned on the same plane (e.g., side-by side (e.g., parallel) or angled relative to each other) on the substrate. For example, co-planar electrodes may include a suitable spacing therebetween and/or include a dielectric material or insulation material disposed between the conducting layers/electrodes. Furthermore, in certain embodiments, one or more of the electrodes may be disposed on opposing sides of the substrate. In such embodiments, contact pads may be one the same or different sides of the substrate. For example, an electrode may be on a first side and its respective contact may be on a second side, e.g., a trace connecting the electrode and the contact may traverse through the substrate.

The sensing layer may be described as the active chemical area of the biosensor. The sensing layer formulation, which can include a glucose-transducing agent, may include, for example, among other constituents, a redox mediator, such as, for example, a hydrogen peroxide or a transition metal complex, such as a ruthenium-containing complex or an osmium-containing complex, and an analyte-responsive enzyme, such as, for example, a glucose-responsive enzyme (e.g., glucose oxidase, glucose dehydrogenase, etc.) or lactate-responsive enzyme (e.g., lactate oxidase). In certain embodiments, the sensing layer includes glucose oxidase. The sensing layer may also include other optional components, such as, for example, a polymer and a bi-functional, short-chain, epoxide cross-linker, such as polyethylene glycol (PEG).

In certain instances, the analyte-responsive enzyme is distributed throughout the sensing layer. For example, the analyte-responsive enzyme may be distributed uniformly throughout the sensing layer, such that the concentration of the analyte-responsive enzyme is substantially the same throughout the sensing layer. In some cases, the sensing layer may have a homogeneous distribution of the analyte-responsive enzyme. In certain embodiments, the redox mediator is distributed throughout the sensing layer. For example, the redox mediator may be distributed uniformly throughout the sensing layer, such that the concentration of the redox mediator is substantially the same throughout the sensing layer. In some cases, the sensing layer may have a homogeneous distribution of the redox mediator. In certain embodiments, both the analyte-responsive enzyme and the redox mediator are distributed uniformly throughout the sensing layer, as described above.

As noted above, analyte sensors may include an analyte-responsive enzyme to provide a sensing component or sensing layer. Some analytes, such as oxygen, can be directly electrooxidized or electroreduced on a sensor, and more specifically at least on a working electrode of a sensor. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing layer proximate to or on a surface of a working electrode. In many embodiments, a sensing layer is formed near or on only a small portion of at least a working electrode.

The sensing layer includes one or more components constructed to facilitate the electrochemical oxidation or reduction of the analyte. The sensing layer may include, for example, a catalyst to catalyze a reaction of the analyte and produce a response at the working electrode, an electron transfer agent to transfer electrons between the analyte and the working electrode (or other component), or both.

A variety of different sensing layer configurations may be used. In certain embodiments, the sensing layer is deposited on the conductive material of a working electrode. The sensing layer may extend beyond the conductive material of the working electrode. In some cases, the sensing layer may also extend over other electrodes, e.g., over the counter electrode and/or reference electrode (or counter/reference is provided).

A sensing layer that is in direct contact with the working electrode may contain an electron transfer agent to transfer electrons directly or indirectly between the analyte and the working electrode, and/or a catalyst to facilitate a reaction of the analyte. For example, a glucose, lactate, or oxygen electrode may be formed having a sensing layer which contains a catalyst, including glucose oxidase, glucose dehydrogenase, lactate oxidase, or laccase, respectively, and an electron transfer agent that facilitates the electrooxidation of the glucose, lactate, or oxygen, respectively.

In other embodiments the sensing layer is not deposited directly on the working electrode. Instead, the sensing layer may be spaced apart from the working electrode, and separated from the working electrode, e.g., by a separation layer. A separation layer may include one or more membranes or films or a physical distance. In addition to separating the working electrode from the sensing layer, the separation layer may also act as a mass transport limiting layer and/or an interferent eliminating layer and/or a biocompatible layer.

In certain embodiments which include more than one working electrode, one or more of the working electrodes may not have a corresponding sensing layer, or may have a sensing layer which does not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing layers by, for example, subtracting the signal.

In certain embodiments, the sensing layer includes one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes including ferrocene. Examples of inorganic redox species are hexacyanoferrate (III), ruthenium hexamine, etc. Additional examples include those described in U.S. Pat. Nos. 6,736,957, 7,501,053 and 7,754,093, the disclosures of each of which are incorporated herein by reference in their entirety.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

Embodiments of polymeric electron transfer agents may contain a redox species covalently bound in a polymeric composition. An example of this type of mediator is poly (vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer including quaternized poly (4-vinyl pyridine) or poly (1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly (1-vinyl imidazole) or poly (4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, including 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly (1-vinyl imidazole) (referred to as “PVI”) and poly (4-vinyl pyridine) (referred to as “PVP”). Suitable copolymer substituents of poly (1-vinyl imidazole) include acrylonitrile, acrylamide, and substituted or quaternized N-vinyl imidazole, e.g., electron transfer agents with osmium complexed to a polymer or copolymer of poly (1-vinyl imidazole).

Embodiments may employ electron transfer agents having a redox potential ranging from about −200 mV to about +200 mV versus the standard calomel electrode (SCE). The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, including a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent, which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

In certain embodiments, the sensor operates at a low oxidizing potential, e.g., a potential of about +40 mV vs. Ag/AgCl. This sensing layer uses, for example, an osmium (Os)-based mediator constructed for low potential operation. Accordingly, in certain embodiments the sensing element is a redox active component that includes (1) osmium-based mediator molecules that include (bidente) ligands, and (2) glucose oxidase enzyme molecules. These two constituents are combined together in the sensing layer of the sensor.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating functions, etc.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly (ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over an enzyme-containing sensing layer and allowing the solution to cure for about one to two days or other appropriate time period. The crosslinker-polymer solution may be applied to the sensing layer by placing a droplet or droplets of the membrane solution on the sensor, by dipping the sensor into the membrane solution, by spraying the membrane solution on the sensor, and the like. Generally, the thickness of the membrane is controlled by the concentration of the membrane solution, by the number of droplets of the membrane solution applied, by the number of times the sensor is dipped in the membrane solution, by the volume of membrane solution sprayed on the sensor, or by any combination of these factors. A membrane applied in this manner may have any combination of the following functions: (1) mass transport limitation, i.e., reduction of the flux of analyte that can reach the sensing layer, (2) biocompatibility enhancement, or (3) interferent reduction.

In some instances, the membrane may form one or more bonds with the sensing layer. By bonds is meant any type of an interaction between atoms or molecules that allows chemical compounds to form associations with each other, such as, but not limited to, covalent bonds, ionic bonds, dipole-dipole interactions, hydrogen bonds, London dispersion forces, and the like. For example, in situ polymerization of the membrane can form crosslinks between the polymers of the membrane and the polymers in the sensing layer. In certain embodiments, crosslinking of the membrane to the sensing layer facilitates a reduction in the occurrence of delamination of the membrane from the sensing layer.

In certain embodiments, the sensing system detects hydrogen peroxide to infer glucose levels. For example, a hydrogen peroxide-detecting sensor may be constructed in which a sensing layer includes enzyme such as glucose oxides, glucose dehydrogenase, or the like, and is positioned proximate to the working electrode. The sensing layer may be covered by one or more layers, e.g., a membrane that is selectively permeable to glucose. Once the glucose passes through the membrane, it is oxidized by the enzyme and reduced glucose oxidase can then be oxidized by reacting with molecular oxygen to produce hydrogen peroxide.

Certain embodiments include a hydrogen peroxide-detecting sensor constructed from a sensing layer prepared by combining together, for example: (1) a redox mediator having a transition metal complex including an Os polypyridyl complex with oxidation potentials of about +200 mV vs. SCE, and (2) periodate oxidized horseradish peroxidase (HRP). Such a sensor functions in a reductive mode; the working electrode is controlled at a potential negative to that of the Os complex, resulting in mediated reduction of hydrogen peroxide through the HRP catalyst.

In another example, a potentiometric sensor can be constructed as follows. A glucose-sensing layer is constructed by combining together (1) a redox mediator having a transition metal complex including Os polypyridyl complexes with oxidation potentials from about −200 mV to +200 mV vs. SCE, and (2) glucose oxidase. This sensor can then be used in a potentiometric mode, by exposing the sensor to a glucose containing solution, under conditions of zero current flow, and allowing the ratio of reduced/oxidized Os to reach an equilibrium value. The reduced/oxidized Os ratio varies in a reproducible way with the glucose concentration, and will cause the electrode's potential to vary in a similar way.

The substrate may be formed using a variety of non-conducting materials, including, for example, polymeric or plastic materials and ceramic materials. Suitable materials for a particular sensor may be determined, at least in part, based on the desired use of the sensor and properties of the materials.

In some embodiments, the substrate is flexible. For example, if the sensor is configured for implantation into a user, then the sensor may be made flexible (although rigid sensors may also be used for implantable sensors) to reduce pain to the user and damage to the tissue caused by the implantation of and/or the wearing of the sensor. A flexible substrate often increases the user's comfort and allows a wider range of activities. Suitable materials for a flexible substrate include, for example, non-conducting plastic or polymeric materials and other non-conducting, flexible, deformable materials. Examples of useful plastic or polymeric materials include thermoplastics such as polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate).

In other embodiments, the sensors are made using a relatively rigid substrate to, for example, provide structural support against bending or breaking. Examples of rigid materials that may be used as the substrate include poorly conducting ceramics, such as aluminum oxide and silicon dioxide. An implantable sensor having a rigid substrate may have a sharp point and/or a sharp edge to aid in implantation of a sensor without an additional insertion device.

It will be appreciated that for many sensors and sensor applications, both rigid and flexible sensors will operate adequately. The flexibility of the sensor may also be controlled and varied along a continuum by changing, for example, the composition and/or thickness of the substrate.

In addition to considerations regarding flexibility, it is often desirable that implantable sensors should have a substrate which is physiologically harmless, for example, a substrate approved by a regulatory agency or private institution for in vivo use.

The sensor may include optional features to facilitate insertion of an implantable sensor. For example, the sensor may be pointed at the tip to ease insertion. In addition, the sensor may include a barb which assists in anchoring the sensor within the tissue of the user during operation of the sensor. However, the barb is typically small enough so that little damage is caused to the subcutaneous tissue when the sensor is removed for replacement.

### Insertion Device

An insertion device can be used to subcutaneously insert the sensor into the user. The insertion device is typically formed using structurally rigid materials, such as metal or rigid plastic. Materials may include stainless steel and ABS (acrylonitrile-butadiene-styrene) plastic. In some embodiments, the insertion device is pointed and/or sharp at the tip to facilitate penetration of the skin of the user. A sharp, thin insertion device may reduce pain felt by the user upon insertion of the sensor. In other embodiments, the tip of the insertion device has other shapes, including a blunt or flat shape. These embodiments may be useful when the insertion device does not penetrate the skin but rather serves as a structural support for the sensor as the sensor is pushed into the skin.

### Sensor Control Unit

The sensor control unit can be integrated in the sensor, part or all of which is subcutaneously implanted or it can be configured to be placed on the skin of a user. The sensor control unit is optionally formed in a shape that is comfortable to the user and which may permit concealment, for example, under a user's clothing. The thigh, leg, upper arm, shoulder, or abdomen are convenient parts of the user's body for placement of the sensor control unit to maintain concealment. However, the sensor control unit may be positioned on other portions of the user's body. One embodiment of the sensor control unit has a thin, oval shape to enhance concealment. However, other shapes and sizes may be used.

The particular profile, as well as the height, width, length, weight, and volume of the sensor control unit may vary and depends, at least in part, on the components and associated functions included in the sensor control unit. In general, the sensor control unit includes a housing typically formed as a single integral unit that rests on the skin of the user. The housing typically contains most or all of the electronic components of the sensor control unit.

The housing of the sensor control unit may be formed using a variety of materials, including, for example, plastic and polymeric materials, such as rigid thermoplastics and engineering thermoplastics. Suitable materials include, for example, polyvinyl chloride, polyethylene, polypropylene, polystyrene, ABS polymers, and copolymers thereof. The housing of the sensor control unit may be formed using a variety of techniques including, for example, injection molding, compression molding, casting, and other molding methods. Hollow or recessed regions may be formed in the housing of the sensor control unit. The electronic components of the sensor control unit and/or other items, including a battery or a speaker for an audible alarm, may be placed in the hollow or recessed areas.

The sensor control unit is typically attached to the skin of the user, for example, by adhering the sensor control unit directly to the skin of the user with an adhesive provided on at least a portion of the housing of the sensor control unit which contacts the skin or by suturing the sensor control unit to the skin through suture openings in the sensor control unit.

When positioned on the skin of a user, the sensor and the electronic components within the sensor control unit are coupled via conductive contacts. The one or more working electrodes, counter electrode (or counter/reference electrode), optional reference electrode, and optional temperature probe are attached to individual conductive contacts. For example, the conductive contacts are provided on the interior of the sensor control unit. Other embodiments of the sensor control unit have the conductive contacts disposed on the exterior of the housing. The placement of the conductive contacts is such that they are in contact with the contact pads on the sensor when the sensor is properly positioned within the sensor control unit.

### Sensor Control Unit Electronics

The sensor control unit also typically includes at least a portion of the electronic components that operate the sensor and the analyte monitoring device system. The electronic components of the sensor control unit typically include a power supply for operating the sensor control unit and the sensor, a sensor circuit for obtaining signals from and operating the sensor, a measurement circuit that converts sensor signals to a desired format, and a processing circuit that, at minimum, obtains signals from the sensor circuit and/or measurement circuit and provides the signals to an optional transmitter. In some embodiments, the processing circuit may also partially or completely evaluate the signals from the sensor and convey the resulting data to the optional transmitter and/or activate an optional alarm system if the analyte level exceeds a threshold. The processing circuit often includes digital logic circuitry.

The sensor control unit may optionally contain a transmitter for transmitting the sensor signals or processed data from the processing circuit to a receiver/display unit; a data storage unit for temporarily or permanently storing data from the processing circuit; a temperature probe circuit for receiving signals from and operating a temperature probe; a reference voltage generator for providing a reference voltage for comparison with sensor-generated signals; and/or a watchdog circuit that monitors the operation of the electronic components in the sensor control unit.

Moreover, the sensor control unit may also include digital and/or analog components utilizing semiconductor devices, including transistors. To operate these semiconductor devices, the sensor control unit may include other components including, for example, a bias control generator to correctly bias analog and digital semiconductor devices, an oscillator to provide a clock signal, and a digital logic and timing component to provide timing signals and logic operations for the digital components of the circuit.

As an example of the operation of these components, the sensor circuit and the optional temperature probe circuit provide raw signals from the sensor to the measurement circuit. The measurement circuit converts the raw signals to a desired format, using for example, a current-to-voltage converter, current-to-frequency converter, and/or a binary counter or other indicator that produces a signal proportional to the absolute value of the raw signal. This may be used, for example, to convert the raw signal to a format that can be used by digital logic circuits. The processing circuit may then, optionally, evaluate the data and provide commands to operate the electronics.

### Calibration

Sensors may be configured to require no system calibration or no user calibration. For example, a sensor may be factory calibrated and need not require further calibrating. In certain embodiments, calibration may be required, but may be done without user intervention, i.e., may be automatic. In those embodiments in which calibration by the user is required, the calibration may be according to a predetermined schedule or may be dynamic, i.e., the time for which may be determined by the system on a real-time basis according to various factors, including, but not limited to, glucose concentration and/or temperature and/or rate of change of glucose, etc.

In addition to a transmitter, an optional receiver may be included in the sensor control unit. In some cases, the transmitter is a transceiver, operating as both a transmitter and a receiver. The receiver may be used to receive calibration data for the sensor. The calibration data may be used by the processing circuit to correct signals from the sensor. This calibration data may be transmitted by the receiver/display unit or from some other source such as a control unit in a doctor's office. In addition, the optional receiver may be used to receive a signal from the receiver/display units to direct the transmitter, for example, to change frequencies or frequency bands, to activate or deactivate the optional alarm system and/or to direct the transmitter to transmit at a higher rate.

Calibration data may be obtained in a variety of ways. For instance, the calibration data may be factory-determined calibration measurements which can be input into the sensor control unit using the receiver or may alternatively be stored in a calibration data storage unit within the sensor control unit itself (in which case a receiver may not be needed). The calibration data storage unit may be, for example, a readable or readable/writeable memory circuit.

Calibration may be accomplished using an in vitro test strip (or other reference), e.g., a small sample test strip such as a test strip that requires less than about 1 microliter of sample (for example FreeStyle® blood glucose monitoring test strips from Abbott Diabetes Care Inc., Alameda, CA). For example, test strips that require less than about 1 nanoliter of sample may be used. In certain embodiments, a sensor may be calibrated using only one sample of body fluid per calibration event. For example, a user need only lance a body part one time to obtain a sample for a calibration event (e.g., for a test strip), or may lance more than one time within a short period of time if an insufficient volume of sample is firstly obtained. Embodiments include obtaining and using multiple samples of body fluid for a given calibration event, where glucose values of each sample are substantially similar. Data obtained from a given calibration event may be used independently to calibrate or combined with data obtained from previous calibration events, e.g., averaged including weighted averaged, etc., to calibrate. In certain embodiments, a system need only be calibrated once by a user, where recalibration of the system is not required.

Alternative or additional calibration data may be provided based on tests performed by a health care professional or by the user. For example, it is common for diabetic individuals to determine their own blood glucose concentration using commercially available testing kits. The results of this test is input into the sensor control unit either directly, if an appropriate input device (e.g., a keypad, an optical signal receiver, or a port for connection to a keypad or computer) is incorporated in the sensor control unit, or indirectly by inputting the calibration data into the receiver/display unit and transmitting the calibration data to the sensor control unit.

Other methods of independently determining analyte levels may also be used to obtain calibration data. This type of calibration data may supplant or supplement factory-determined calibration values.

In some embodiments of the invention, calibration data may be required at periodic intervals, for example, every eight hours, once a day, or once a week, to confirm that accurate analyte levels are being reported. Calibration may also be required each time a new sensor is implanted or if the sensor exceeds a threshold minimum or maximum value or if the rate of change in the sensor signal exceeds a threshold value. In some cases, it may be necessary to wait a period of time after the implantation of the sensor before calibrating to allow the sensor to achieve equilibrium. In some embodiments, the sensor is calibrated only after it has been inserted. In other embodiments, no calibration of the sensor is needed.

### Analyte Monitoring Device

In some embodiments of the invention, the analyte monitoring device includes a sensor control unit and a sensor. In these embodiments, the processing circuit of the sensor control unit is able to determine a level of the analyte and activate an alarm system if the analyte level exceeds a threshold value. The sensor control unit, in these embodiments, has an alarm system and may also include a display, such as an LCD or LED display.

A threshold value is exceeded if the datapoint has a value that is beyond the threshold value in a direction indicating a particular condition. For example, a datapoint which correlates to a glucose level of 200 mg/dL exceeds a threshold value for hyperglycemia of 180 mg/dL, because the datapoint indicates that the user has entered a hyperglycemic state. As another example, a datapoint which correlates to a glucose level of 65 mg/dL exceeds a threshold value for hypoglycemia of 70 mg/dL because the datapoint indicates that the user is hypoglycemic as defined by the threshold value. However, a datapoint which correlates to a glucose level of 75 mg/dL would not exceed the same threshold value for hypoglycemia because the datapoint does not indicate that particular condition as defined by the chosen threshold value.

An alarm may also be activated if the sensor readings indicate a value that is outside of (e.g., above or below) a measurement range of the sensor. For glucose, the physiologically relevant measurement range is typically 30-400 mg/dL, including 40-300 mg/dL and 50-250 mg/dL, of glucose in the interstitial fluid.

The alarm system may also, or alternatively, be activated when the rate of change or acceleration of the rate of change in analyte level increase or decrease reaches or exceeds a threshold rate or acceleration. For example, in the case of a subcutaneous glucose monitor, the alarm system may be activated if the rate of change in glucose concentration exceeds a threshold value which may indicate that a hyperglycemic or hypoglycemic condition is likely to occur. In some cases, the alarm system is activated if the acceleration of the rate of change in glucose concentration exceeds a threshold value which may indicate that a hyperglycemic or hypoglycemic condition is likely to occur.

A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

### Drug Delivery System

The subject invention also includes sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit such as a transmitter, a receiver/display unit, and a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Analyte Sensor and Inflammation

Tissue reactions at sites of glucose sensor implantation, e.g. inflammation and fibrosis, are generally thought to be contributors to the loss of glucose sensor function in vivo following sensor insertion in a subject.

Cytokines are small molecular weight glycoproteins (e.g., <20,000 MW) that play a role in controlling innate and acquired immunity, inflammation and wound healing (e.g., angiogenesis, regeneration and fibrosis) in a wide variety of diseases and infections. Among the various cytokine families involved in inflammation and wound healing, the Interleukin 1 (IL-1) and tumor necrosis factor (TNF) families appear to be major inflammatory networks. For example, IL-1 Beta (IL-1B) and TNFalpha (TNFa) are considered to be initiators of a wide range of pro-inflammatory cell and tissue reactions (i.e., prime-cytokines). These cytokines play a role in immunity and host defense, as well as acute and chronic inflammatory diseases such as rheumatoid arthritis, inflammatory bowel disease and interstitial lung disease.

Interleukin 1Beta (IL-1B) is a pro-inflammatory cytokine and its regulation prevents uncontrolled inflammation and tissue destruction including foreign body reactions. The IL-1B antagonist, IL-1RN, plays a role in controlling IL-1B mediated inflammation. IL-1RN competes with IL-1 for binding to the IL-1 receptors, and thereby prevents IL-1 activation of both leukocytes and tissue cells. The role of IL-1RN in controlling inflammation has also been supported by studies using transgenic mice that demonstrate that over-expression of IL-1RN in these mice suppresses inflammation, and IL-1RN knockout mice have increased inflammation and tissue destruction.

Currently available glucose sensors for human use are approved for an implantation period generally of 7 days or less. Developing a better understanding of the role of the cells, factors and tissue reactions (e.g., the effect of the foreign body tissue reaction at the implantation site) that occur at sites of sensor implantation and their relationship to sensor function may provide better rationales and approaches to extending glucose sensor function in vivo. To investigate the role of IL-1 in glucose sensor function in vivo, sensor function was compared in transgenic mice that 1) over-express IL-1RN (B6.Cg-Tg (IL1rn)1Dih/J) and 2) are deficient in IL-1RN (B6.129S-Il1 rn^{tm1Dih}/J) with mice that have normal levels of IL-1RN (C57BL/6). These studies indicated that 1) IL-1 family of cytokines, likely IL-1B, play a role in controlling tissue reactions and sensor function in vivo, and 2) the IL-1 antagonist IL-1RN plays a role in controlling tissue reactions and sensor function in vivo. These studies suggested that targeting the IL-1 family of cytokines, e.g., local delivery of IL-1 antagonists at sites of sensor implantation may enhance short-term sensor function in vivo and possible long-term sensor function in vivo.

Interleukin 1 cytokine Family and Inflammation

Cytokines are low molecular weight glycoproteins secreted by tissue, inflammatory, and tumor cells, which can regulate cell functions in an autocrine or paracrine fashion. The cytokine interleukin-1 (IL-1) is a regulator of inflammation and immune response. IL-1 is a multifunctional cytokine able to affect virtually all cell types. The IL-1 family consists of two agonists, IL-1a and IL-1B, a competitive antagonist, IL-1 receptor antagonist (IL-1RN/IL-1RA), and two receptors IL-1RI and IL-1RII. IL-1a and IL-1B show approximately 25% amino acid homology. IL-1a is the acidic form while IL-1B is the neutral form. Both IL-1a and IL-1B are synthesized as 31 kDa precursors, which are cleaved into 17 kDa proteins. These cytokines lack classical signal peptides (for secretion) yet IL-1a and IL-1B exert their physiological effects by binding to specific receptors. While IL-1a remains intracellular and is released upon cell death, IL-1B is secreted out of the cell. IL-1 is a potent inducer of inflammation and, unlike other cytokines, IL-1-mediated cellular activation is regulated at multiple levels. Control of an inflammatory event may depend on the concentration of the interleukin-1 antagonist, IL-1RN and the ratio of IL-1RN/IL-1 within the tissue microenvironment. IL-1RN competes for binding to the IL-1Rs and thereby prevents IL-1 from activating the receptor. Isoforms of IL-1RN have been identified and include: one secreted form (sIL-1RN) and three intracellular forms (icIL1RN 1, 2, and 3). While sIL-1RN competitively inhibits IL-1 receptor binding, icIL1ra may not only inhibit IL-1 binding, but also regulate IL-1 responses beyond the receptor level. IL-1RI is a 80 kDa membrane bound receptor while IL-1RII is a 68 kDa protein, but both are members of the immunoglobulin superfamily. The two receptors share 28% homology in their extracellular domains but differ in their cytoplasmic regions. Where IL-1RI has a 213 amino acid cytoplasmic domain, IL-1RII contains only 29 amino acids in this region. IL-1RI is the signal transducing receptor and IL-1RII does not transduce a signal when IL-1 is bound to it and is considered an IL-1 ‘sink’. Additionally, IL-1RII exists not only as a membrane bound form, but can also be found as a soluble form in the circulation of healthy adults. Therefore, IL-1RI mediates IL-1 signal transduction and IL-1RII is involved in down-regulation or inhibition of IL-1 activation. IL-1 activation may require that IL-1/IL-1RI complex associate with interleukin-1 receptor accessory protein (IL-1RacP) to mediate signal transduction. The mechanism by which IL-1 mediates its activity is via activation of the inhibitor of κB/nuclear factor-κB (IκB/NFκB) and AP-1 transcription factor pathways. NFκB has been shown or implicated in the regulation of a number of protumorogenic activities including: a) regulation of invasiveness/metastasis factors such as metalloproteinase (MMP), urokinase plasminogen activator (uPA), and endothelial cell adhesion molecules (selectins) critical for angiogenesis; and b) a number angiogenic/mitogenic cytokines such as growth-regulated oncogene protein (GRO), IL-8, vascular endothelial growth factor (VEGF), basic fibroblast growth factor (bFGF) and tumor necrosis factor (TNF) as well as the motility factor, IL-6.

### Methods and Materials

The following methods and materials were used in the Examples below.

### IL-1RN Knockout and IL-1RN Over-Expression Mouse Models

For the present in vivo studies, female IL-1RN Knockout mice (IL-1RN-KO) and IL-1RN Over-Expressing mice (IL-1RN-OE) were utilized. IL-1RN Knockout (B6.129S-Il1rn^{tm1Dih}/J) and IL-1RN Over-Expressing mice (B6.Cg-Tg (II1rn)1Dih/J) were obtained from Jackson Laboratory (Bar Harbor, Maine). All mice were maintained on antibiotic water for the duration of the experiment. Additionally, Female C57BL/6 mice were used as normal controls for these studies, and were also obtained from Jackson Laboratory.

### Glucose Sensors, Implantation and Murine Continuous Glucose Sensor System

All modified Navigator™ glucose sensors used in these in vivo studies were obtained from Abbott Diabetes Care. Sensor were modified by removal from the standard transdermal insertion unit, and by the attachment of wires to the electrode contact pads. Glucose sensors were implanted into IL-1RN-KO, IL-1RN-OE or C57BL/6 mice and continuous glucose monitoring (CGM) was undertaken for a period of 7 days as described (Klueh et al., Biomaterials 2010; 31 (16): 4540-51, Klueh et al., Diabetes Technol Ther 2006; 8 (3): 402-12). For the present studies, all sensor data was presented as raw current signals (nA) in order to evaluate the true non-calibrated signal dynamics, i.e., no sensor calibration or recalibration. Current data at 60-second intervals, were overlaid on blood glucose reference measurements in dual y-axis plots, to obtain a best visual fit. Blood glucose reference measurements were obtained at least daily using blood obtained from the tail vein of the mouse and a FreeStyle® Blood Glucose Monitor. The Institutional Animal Care and Use Committee of the University of Connecticut Health Center (Farmington, CT) approved all mice studies.

### Histopathologic Analysis of Tissue Reactions at Glucose Sensor Implantation Sites

In order to evaluate tissue responses to glucose sensor implantation at various time points, individual mice were euthanized and the tissue containing the implanted sensors were removed, fixed in Zinc buffer for 24 hours, followed by standard processing, embedded in paraffin and sectioned. The resulting 4-6 um sections were then stained using standard protocols for H&E and Masson Trichrome (fibrosis). Histopathologic evaluation of tissue reactions at sites of sensor implantation was performed on mouse specimens obtained at 1, 3, and 7 days post implantation (DPI) of the glucose sensor. The tissue samples were examined for signs of inflammation, including necrosis, fibrosis, angiogenesis, and vessel regression. Resulting tissue sections were evaluated directly and documented by digitized imaging using an Olympus Digital Microscope.

### Example 1

### Glucose Sensor Function in Normal Mice (C57BL/6)

### Continuous Glucose Monitoring in Normal Mice

Tissue responses to an implanted sensor may become increasingly more important as the implantation period is increased. In order to achieve long-term glucose sensing, the severity of the tissue reaction occurring in the initial phase of sensor implantation (e.g., tissue injury) may have an impact on the tissue repair at site of sensor implantation. Therefore, experiments were performed to study potential mediators and mechanisms that control sensor related tissue reactions within the first 7 days post implantation. A murine model of continuous glucose monitoring (CGM) was used. Since the Interleukin 1 family of cytokines mediates inflammation and repair, the role of IL-1/IL-1RN in glucose sensing was investigated using genetically engineered mice, which lack IL-1RN (e.g., IL-1RN-KO knockout mice) or over-express IL-1RN (e.g., IL-1RN-OE mice). Experiments were also performed on CGM in normal C57BL/6 mice. CGM during the first 7 days resulted in a sensor output that closely paralleled blood glucose levels monitored externally (FIGS. 1A-D). The glucose sensor consistently detected both hyperglycemic and hypoglycemic events during the 7 days of CGM (FIGS. 1A-D). These results were used for comparison of CGM in IL-1RN-KO and IL-1RN-OE mice described below.

The CGM profile of normal C57BL/6 mice was determined over a 7-day post sensor implantation time period (FIGS. 1A-H). FIG. 1E represents the magnified view of FIG. 1A; FIG. 1F represents the magnified view of FIG. 1B; FIG. 1G represents the magnified view of FIG. 1C and FIG. 1H represents the magnified view of FIG. 1D. The glucose sensors displayed accurate CGM during the first 7 days post implantation with glucose sensing closely following highs and lows of mouse blood glucose levels (FIGS. 1A-D). Data presented in FIGS. 1A, 1B and 1C show an increase in the blood glucose level around day 2 post sensor implantation. In FIG. 1A (including the magnified view in FIG. 1E), this increase is not obvious but it is theorized that since the mouse had a low blood sugar level (around 50 mg/dL) for a significant time period, the mouse started eating and developed a more physiological blood glucose level after the initial implantation period. The apparent blood sugar level in FIG. 1B is most likely due to handling the mouse as a result of a cage change. An increased stress level (e.g., cage changes, isoflurane administration, noise, etc.) may temporarily increase the blood sugar level. The blood sugar level of the mouse illustrated in FIG. 1C and FIG. 1G was not in the physiological range and the mouse was provided with a high sugar solution. Therefore, the spike in the mouse blood glucose level is in response of the oral uptake of glucose. The glucose sensor tracked both hyperglycemic and hypoglycemic events in the normal mice (FIGS. 1A-H). These data demonstrated that the glucose sensor had an accurate response profile throughout the first week post implantation and was consistent with previously published data (Klueh et al., Biomaterials 2010; 31 (16): 4540-51, Klueh et al., Diabetes Technol Ther 2006; 8 (3): 402-12).

### Example 2

### Glucose Sensor Function in IL-1RN Knockout Mice

### Continuous Glucose Monitoring in Interleukin 1 Receptor Antagonist Knockout Mice

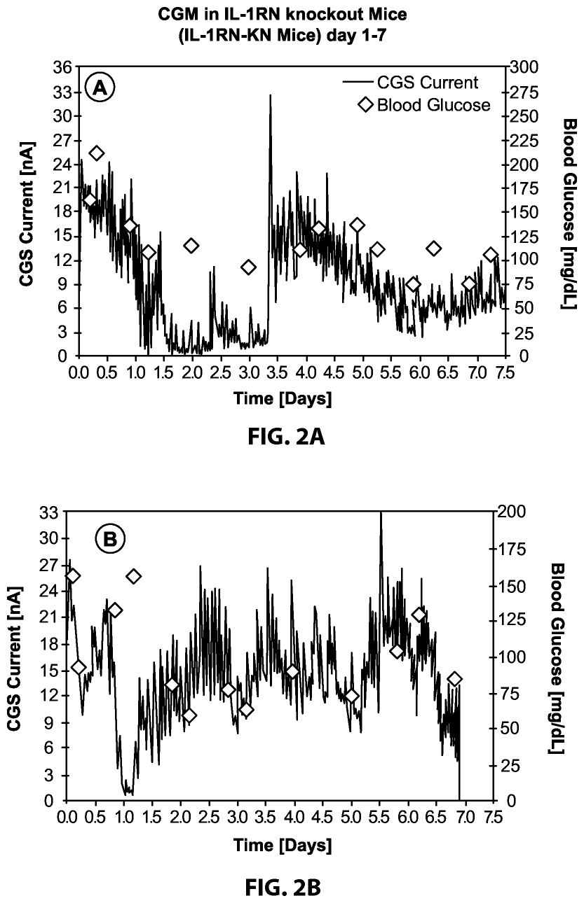

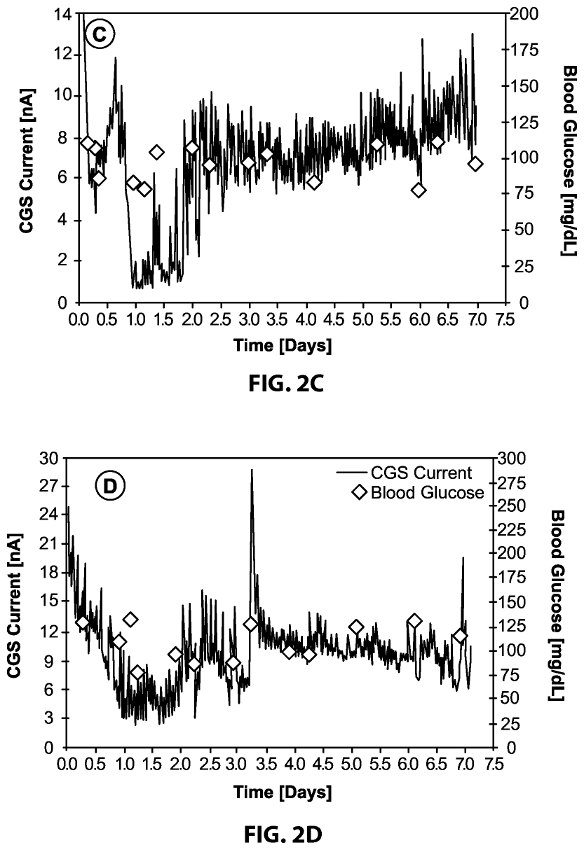

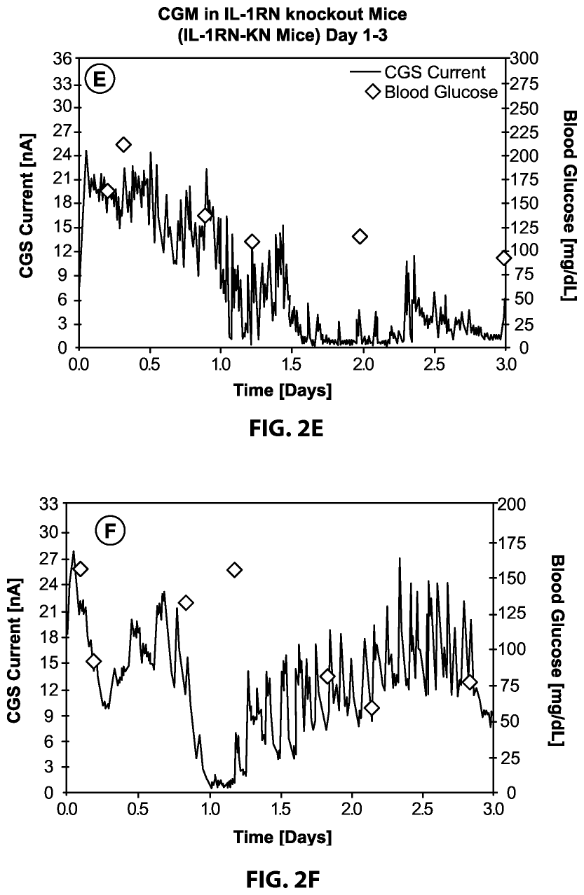

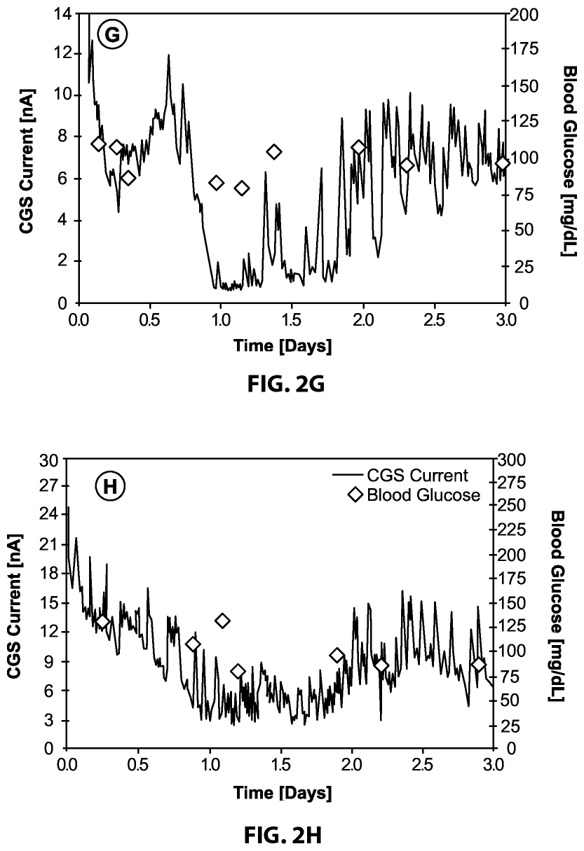

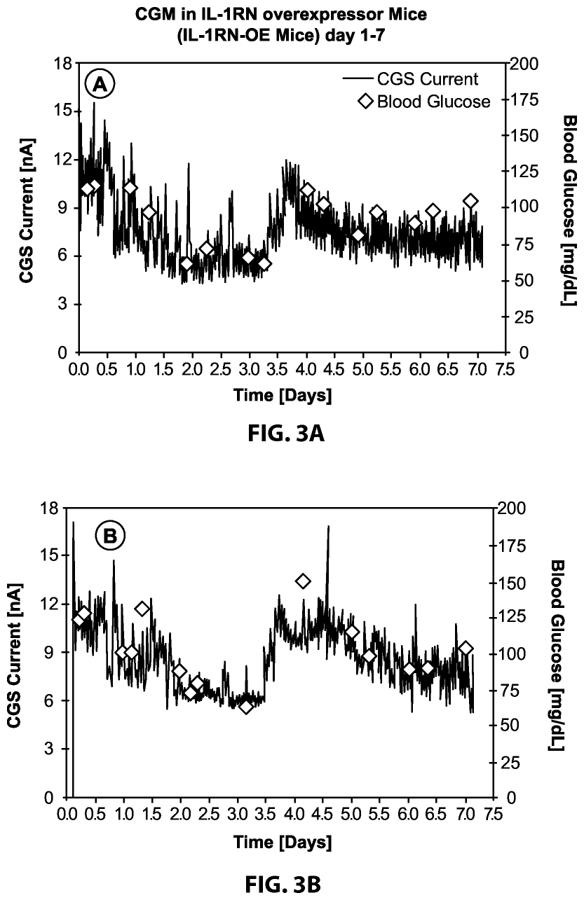

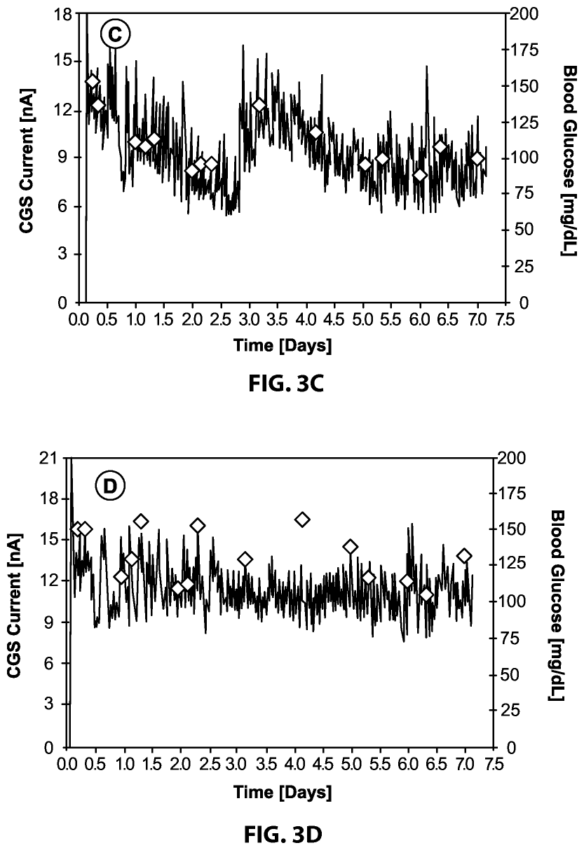

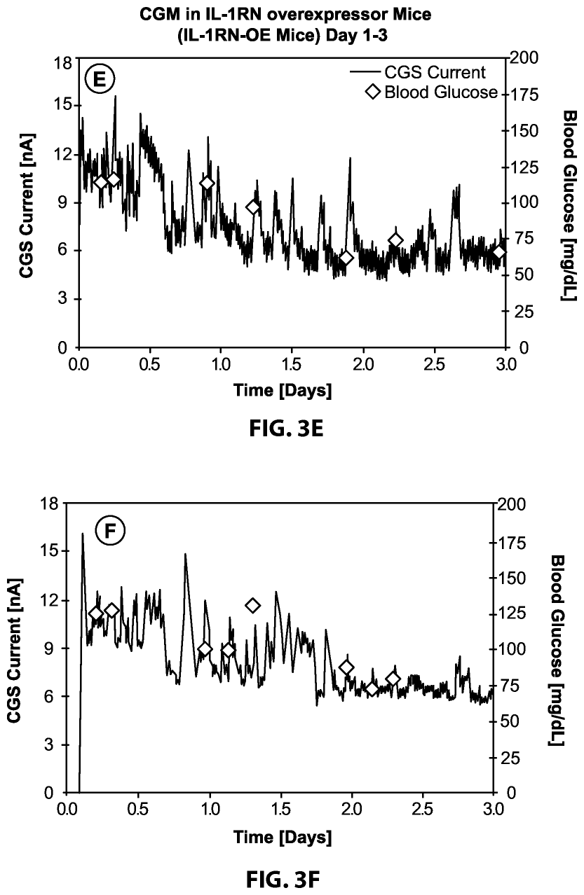

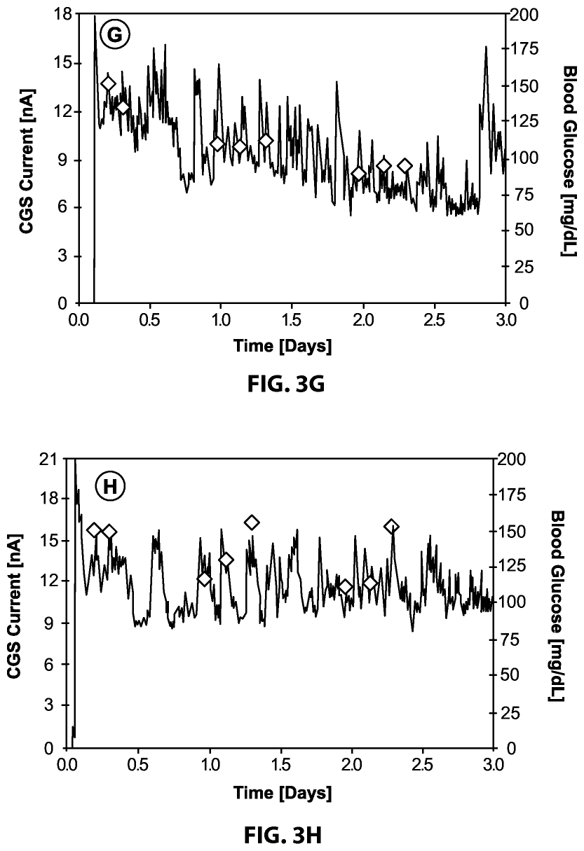

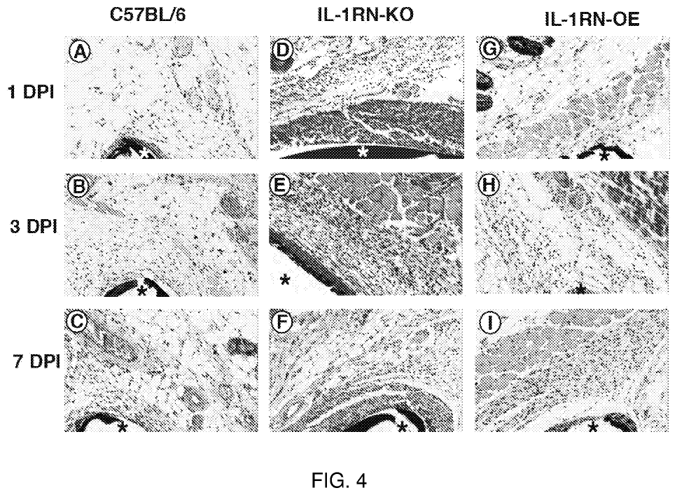

Because of the pro-inflammatory and pro-fibrotic activity of IL-1B, removing IL-1 antagonist, IL-1RN, expression in vivo, may allow over expression of pro-inflammatory activity of locally produced IL-1B, resulting in enhanced inflammation and fibrosis and decreased glucose sensor function. The experiments demonstrated that deficiency of IL-1RN in IL-1RN-KO mice resulted in an increase in inflammation at the site of sensor implantation (FIGS. 3A-H and FIG. 4), which correlated with loss of sensor function within the first few days post sensor implantation (FIG. 2A-H). Sensor functionality was lost typically within the first 24 hours post implantation and in most cases this temporary loss of sensor functionality lasted for the first 2-3 days. The initial implantation of the sensor triggered release of local inflammatory mediators from tissue cells, and plasma proteins resulting from an increased vasopermeability, including leukocytes chemotactic factors (LCF). These locally expressed LCF in turn recruited both polymorphonucelar leukocytes (PMNs) and monocyte/macrophages. Both PMNs and MQs express IL-1, and MQs are a source of locally produced IL-1RN. Additionally, the initial increase in vasopermeability associated with sensor implantation trauma may act to inhibit acute IL-1B activity as well as supplement local MQ expression IL-1RN. Since IL-1RN KO mice were deficient in the antagonist IL-1RN, IL-1 expression was not regulated during this phase of sensor implantation and tissue injury. Therefore, IL-1 expression levels were increased, which had an effect on sensor functionality post sensor implantation typically within the first 24 hours. For example, within the first 24 hours, sensor output declined rapidly and sharply (FIGS. 2B and 2C) or declined continuously over a few hours (FIGS. 2A and 2D). This loss of sensor function typically lasted for 1 day, but may also span over several days before the sensor output increased again and started correlating with the reference blood glucose measurements. This regain of sensor functionality might be attributed to the process of wound healing. During wound repair, new blood vessels were formed to allow the passage of proteins and cells to the site of tissue injury. With the formation of new vessels, better diffusion of the glucose analyte to the sensing layer of the sensor may occur, allowing the sensor output to increase to its initial value.

Since IL-1RN plays a role in controlling tissue reactions at sites of sensor implantation, the effect of IL-1RN deficiency on sensor function was tested using the IL-1RN knockout mice (IL-1RN-KO). Over the 7-day period of CGM, sensor output occasionally failed to reliably track with blood glucose levels in the IL-1RN-KO mice (FIGS. 2A-H). FIG. 2E represents the magnified view of FIG. 2A; FIG. 2F represents the magnified view of FIG. 2B; FIG. 2G represents the magnified view of FIG. 2C and FIG. 2H represents the magnified view of FIG. 2D. For example, sensor output 1-3 days post sensor implantation consistently failed to correlate with blood glucose levels in the IL-1RN-KO mice (FIGS. 2A-H). Additionally, sensor output beyond 3 days post sensor implantation was occasionally inaccurate in the IL-1RN-KO mice (FIG. 2A), but in most cases correlated well with the sporadic blood glucose reference measurements (FIGS. 2B-D). In summary, unlike normal C57BL/6 mice (FIGS. 1A-H), sensor output in IL-1RN knockout mice during 7 days of CGM failed to consistently track hyperglycemic and hypoglycemic events in these mice (FIGS. 2A-H), particularly within the first 72 hours post sensor implantation. These data directly support the hypothesis that IL-1 and IL-1RN play a role in short term CGM in vivo.

### Example 3

### Glucose Sensor Function in IL-1RN Over-Expressing Mice

### Continuous Glucose Monitoring in Interleukin 1 Receptor Antagonist Over-Expressing Mice

CGM experiments that utilized IL-1RN-KO were performed. The experiments showed that IL-1/IL-1RN plays a role in controlling both tissue reactions and glucose sensor function at sites of sensor implantation. Over-expression of IL-1RN may allow blocking of pro-inflammatory activity of locally produced IL-1B, resulting in decreased inflammation and fibrosis and increased glucose sensor function. The experiments demonstrated that over expression of IL-1RN in IL-1RN-OE mice resulted in an increase in inflammation and fibrosis at the site of sensor implantation (FIGS. 3A-H and 4) when compared to the IL-1RN-KO mice (FIGS. 2A-H). For the 7 day testing period, IL-1RN-OE mice displayed similar sensor function as C57BL control mice. These experiments suggested that a decrease in systemic and/or local IL-1RN expression may cause a decrease in sensor function. Alternatively, if an anti-inflammatory agent (e.g., IL-1 inhibitors/antagonists) was locally delivered to the site of sensor implantation, short-term sensor performance and lifespan may be extended.

The IL-1RN-KO studies described above indicated that the absence of IL-1RN decreased glucose sensor function in vivo. To confirm these observations, experiments were performed to study the effect over-expression of IL-1RN had on sensor function using IL-1RN over-expressing mice (IL-1RN-OE). As was the case with C57BL/6 mice, sensor output in IL-1RN-OE mice correlated well with the reference blood glucose measurement during the entire 7-day testing period (FIGS. 3A-H). FIG. 3E represents the magnified view of FIG. 3A; FIG. 3F represents the magnified view of FIG. 3B; FIG. 3G represents the magnified view of FIG. 3C and FIG. 3H represents the magnified view of FIG. 3D. These data demonstrate the role IL-1RN has in controlling IL-1 effects in the initial days post sensor implantation.

### Example 4

### Inflammation and Fibrosis at the Sites of Glucose Sensor Implantation

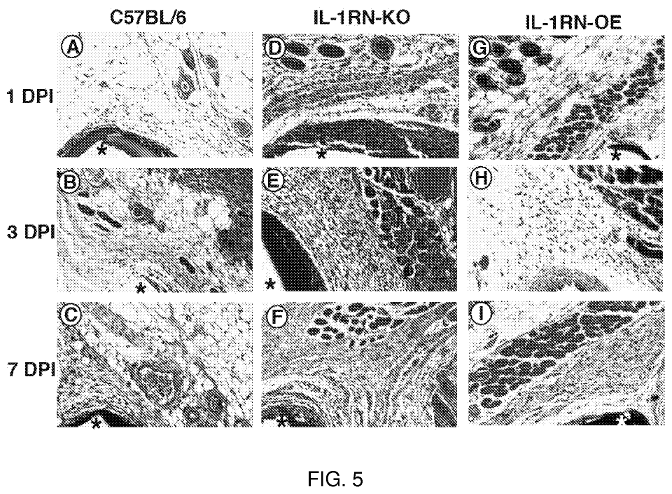

The sensor function in normal, IL-1RN-KO and IL-1RN-OE mice described above demonstrated the role of IL-1/IL-1RN in controlling sensor function in vivo. Experiments were performed to determine how alterations in IL-1RN expression influenced sensor function in vivo. For example, IL-1 may drive inflammation and fibrosis at sites of sensor implantation. Therefore, by removing IL-1RN control of the IL-1 activity (i.e., IL-1RN deficiency/knockout) an increase in inflammation and fibrosis at sites of sensor implantation may occur. Thus, experiments were performed to evaluate sensor tissue sites using H&E as well as trichrome staining technology at 1, 3 and 7 days post sensor implantation. As can be seen in FIG. 4 IL-1RN deficiency increased tissue reactions of inflammation (FIG. 4, Panels D-F) and fibrosis (FIG. 5, Panels D-F) when compared to normal (FIG. 4, Panels A-E and FIG. 5, Panels A-E) or IL-1RN-OE (FIG. 4, Panels G-I and FIG. 5, Panels G-I) mice. For example, inflammation was consistently greater in the IL-1RN-KO mice both at early stages post sensor implantation (PSI) (e.g., 1-3 days post implantation (DPI)) as well as later stages (e.g., 7 days post implantation (DPI)) post sensor implantation (PSI), as compared to normal and IL-1RN-OE mice (FIG. 4, Panels A-I). There was significantly higher macrophage accumulation at the interface of the sensor with tissue in the IL-1RN-KO mice (FIG. 4, Panels D-F), when compared to normal (FIG. 4, Panels A-C) or IL-1RN-OE mice (FIG. 4, Panels G-I). This increase in macrophages (MQ) at the interface of the sensor may be significant since MQ cells control inflammation and fibrosis at sites of tissue injury, including foreign body reactions.

Using Trichrome staining techniques, the effect of IL-1RN deficiency (IL-1RN-KO mice) or overexpression (IL-1RN-OE) mice on fibrosis at the site of sensor implantation was studied. Because of the relatively short time period of 7 days, it was expected that only limited fibrosis could occur at implantations sites. In normal mice (C57B6), there was no significant collagen associated with implanted sensors 1-3 DPI, and by 7 DPI there was only limited collagen association with the implanted sensors (FIG. 5, Panels A-C). In the IL-1RN-OE mice, limited collagen association was also observed with the implanted sensors, at 1-3 DPI and slightly more by 7 days post implantation (DPI) (FIG. 5, Panels G-I). In the case of the IL-1KO mice, there appeared to be slightly higher association between collagen and the implanted sensor, by 7 DPI (FIG. 5, Panels D-F). This collagen-sensor association in the IL-1-KO mice was likely the result of the high level of inflammation seen at the site of sensor implantation in the IL-1-KO mice. The impact of IL-1RN deficiency on inflammation and fibrosis at the tissue sensor interface may negatively affect sensor function in vivo, as seen in the IL-1RN KO mice (FIG. 5, Panels D-F). Since IL-1B has a role in controlling fibroblast function in vivo, the lack of IL-1RNs at the sensor tissue interface in the IL-1/IL-1RN deficient mice may contribute to the decrease in fibrosis for time periods post 1-week sensor implantation. Alternatively, since IL-1 controlled fibroblast function, over-expression of IL-1RN may directly decrease both the recruitment and activation of fibroblasts at site of sensor implantation. Both of the factors may contribute to a decrease in fibrosis seen in IL-1RN over-expressing mice.

### Tissue Reactions to Implanted Glucose Sensors in Normal, IL-1RN Knockout and IL-1RN Over-Expressing Mice

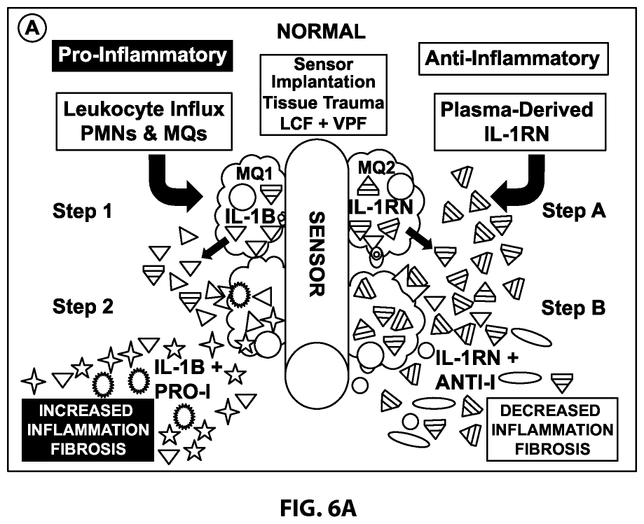

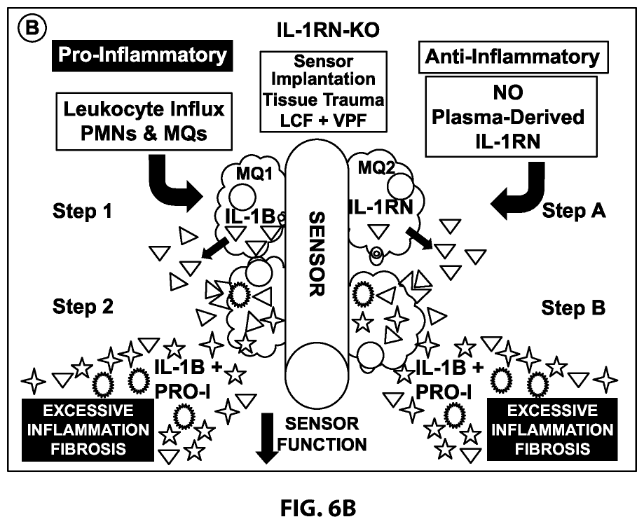

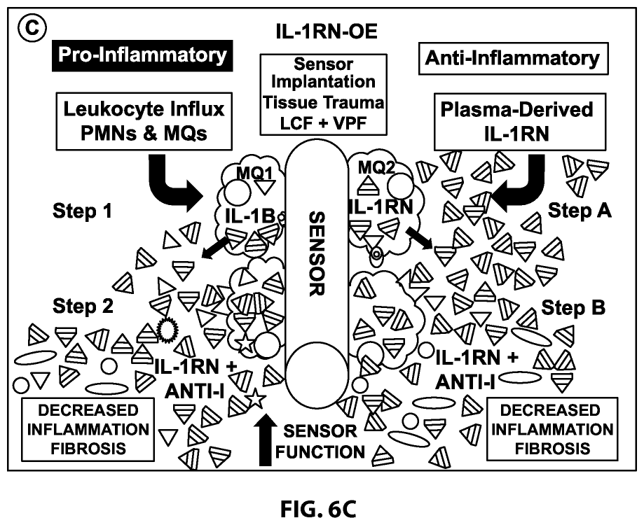

The experimental results indicated that the IL-1 family of cytokines (agonists and antagonists) play a role in controlling tissue reactions and thereby sensor function at sites of glucose sensor implantations. For example, in normal mice (C57B/6) the initial sensor-associated tissue trauma induced both leukocyte accumulation, via local expression of LCFs (FIG. 6A, Step 1), as well as increased vasopermeability (FIG. 6A, Step A), which caused an influx of plasma derived IL-1RN. This initial influx of plasma IL-1RN was adequate to control the initial levels of IL-1B produced at the site of sensor implantation, but not the increased local production of IL-1B by both activated leukocytes (recruited) and tissue cells (FIG. 6A, Step 2), for example from the induction of the MI class of pro-inflammatory macrophages (FIG. 6A, Step 2). This increase in IL-1B expression resulted in induction of other pro-inflammatory cytokines such as IL-6, IL-8, MCP, INFg, which increased the inflammatory reactions at the site of sensor implantation, ultimately leading to a reduction in sensor function (FIG. 6A). This IL-1B expression is likely neutralized by up regulation of IL-1RN expression in M2 Macrophages and activated tissue cells (FIG. 6A, Step B). This IL-1RN based inhibition of IL-1B not only reduced inflammation and tissue injury, both of which enhanced glucose sensor function and life span (FIG. 6A, Step 2).

In the case of the IL-1RN-KO mice, the lack of plasma or cell derived IL-1RN allowed the dominancy of IL-1B pro-inflammatory both at early stages (FIG. 6B, Steps 1 and A) and later stages (FIG. 6B, Steps 2 and B) post sensor implantation. This dominancy of the pro-inflammatory IL-1B expression expanded inflammation and tissue injury by inducing pro-inflammatory macrophages (MI class MQs) and tissue cells which expressed even more pro-inflammatory cytokines such IL-6, IL-8, MCP, INFg, etc., which induced excessive inflammation and tissue destruction, thus reducing sensor function in vivo (FIG. 6B, Steps 2 and B).

Alternatively, in IL-1RN-OE mice the pro-inflammatory actions of IL-1B were limited in both the early (FIG. 6C, Steps 1 and A) and late stages (FIG. 6C, Steps 2 and B) post sensor implantation. For example, the increased expression of IL-1B by both recruited leukocytes and tissue cells plus plasma levels of IL-1RN effectively suppressed the initial IL-1B associated with sensor implantation (FIG. 6C, Steps 1 and A). Additionally, the continued overexpression of IL-1RN by both MQs and tissue cells continued to suppress IL-1B activation of MQs and tissue cells, not only limiting local production of IL-1B from these cells but also other pro-inflammatory cytokines (FIG. 6C, Steps 2 and B). Thus, over-expression of IL-1RN resulted in: 1) a decrease in the expression of IL-1B; 2) a decrease in IL-1B induced pro-inflammatory cytokines; and 3) an increase M2 class anti-inflammatory MQs. The end result of these IL-1RN dependent events was to decrease inflammation and fibrosis, as well as increase neovasculariztion at the site of sensor implantation. The anti-inflammatory effect of IL-1RN over-expression resulted in extended glucose sensor function in vivo.

The experimental results demonstrated that the IL-1 family of cytokines (agonists and antagonists) played a role in controlling tissue reactions and glucose sensor function at sites of sensor implantation, and also demonstrated that the local delivery of IL-1B inhibitors and antagonists (e.g., local delivery of recombinant IL-1RN, IL-1RN gene therapy, antibodies to IL-1B, local delivery of recombinant soluble IL-1 receptors and IL-1 receptor gene therapy) may reduce inflammation and fibrosis and increase glucose sensor function in vivo. The experiments demonstrated that the IL-1 family of cytokines play a role in tissue reactions and sensor function over the initial 7 days post sensor implantation, and that IL-1 and IL-1RN play a role in controlling long-term tissue reactions at sites of sensor implantation, as well as in long-term continuous glucose sensing in vivo.

The results of the studies showed that glucose sensor function was decreased in IL-1RN knockout mice, when compared to IL-1RN over-expressing and normal mice. Additionally, histologic analysis of the various sensor implantation sites indicated that excessive inflammation was associated with sensors in IL-1RN knockout mice, but not in IL-1RN over-expressing or normal mice. The experiments indicated the role the IL-1 family of cytokines play in glucose sensor function and associated tissue reaction, and also showed that local delivery of IL-1 antagonists extended glucose sensor function in vivo, which may be useful in long-term in vivo glucose sensors, for example, in vivo glucose sensors used in long-term closed-loop glucose monitoring systems.

The preceding merely illustrates the principles of embodiments of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An in vivo analyte sensor for measuring analyte levels in a bodily fluid of a user comprising:
a first portion coupled with a sensor control unit, wherein the first portion is positionable above a surface of a skin,
a second portion positionable below the surface of the skin, wherein the second portion is in contact with a bodily fluid and configured to measure signals indicative of analyte levels in the bodily fluid, wherein the second portion comprises electrodes connected to contact portions positioned on the first portion;
wherein the sensor control unit comprises:
a processor configured to determine data indicative of analyte levels and to transmit the data indicative of analyte levels to a receiver unit according to a Bluetooth communication protocol via a transmitter coupled to the processor, and
a power supply for operating the sensor control unit;
further wherein the electrodes of the second portion include a working electrode comprising a sensing layer disposed thereon and a membrane layer disposed at least partially over the sensing layer, wherein the sensing layer comprises an osmium-containing complex, and wherein the membrane layer comprises a polyvinylpyridine;
a substrate; and
a counter electrode,
wherein a polymer layer comprising dexamethasone is disposed on an exterior surface of the working electrode, the substrate, or the counter electrode, the polymer layer being separate from the sensing layer and the membrane layer; and
wherein the sensor control unit is configured to process factory-determined calibrated measurements that are input or stored in the sensor control unit, such that the analyte sensor is thereby factory calibrated.

2. The analyte sensor of claim 1, wherein the polymer layer is disposed on the exterior surface of the counter electrode.

3. The analyte sensor of claim 1, wherein the polymer layer is disposed on the exterior surface of the substrate.

4. The analyte sensor of claim 1, wherein the polymer layer is disposed on the exterior surface by dip coating, spray coating, or drop deposition.

5. The analyte sensor of claim 1, wherein the sensing layer comprises an analyte-responsive enzyme.

6. The analyte sensor of claim 7, wherein the analyte-responsive enzyme is a glucose-responsive enzyme.

7. A method comprising:
detecting signals representative of an in vivo analyte level in a bodily fluid of a user using an analyte sensor comprising:
a first portion coupled with a sensor control unit, wherein the first portion is positionable above a surface of a skin;
a second portion positionable below the surface of the skin, wherein the second portion is in contact with a bodily fluid and configured to measure signals indicative of analyte levels in the bodily fluid, wherein the second portion comprises electrodes connected to contact portions positioned on the first portion;
wherein the sensor control unit comprises
a processor configured to determine date indicative of analyte levels and to transmit the data indicative of analyte levels to a receiver unit according to a Bluetooth communication protocol via a transmitter coupled to the processor, and
a power supply for operating the sensor control unit;
further wherein the electrodes of the second portion include a working electrode comprising a sensing layer disposed thereon and a membrane layer disposed at least partially over the sensing layer, wherein the sensing layer comprises an osmium-containing complex, and wherein the membrane layer comprises a polyvinylpyridine;
a substrate; and
a counter electrode,
wherein a polymer layer comprising dexamethasone is disposed on an exterior surface of the working electrode, the substrate, or the counter electrode, the polymer layer being separate from the sensing layer and the membrane layer; and
wherein the sensor control unit is configured to process factory-determined calibrated measurements that are input or stored in the sensor control unit, such that the analyte sensor is thereby factory calibrated.

8. The method of claim 7, wherein the polymer layer is disposed on the exterior surface of the counter electrode.

9. The method of claim 7, wherein the polymer layer is disposed on the exterior surface of the substrate.

10. The method of claim 7, wherein the polymer layer is disposed on the exterior surface by dip coating, spray coating, or drop deposition.

11. The method of claim 7, wherein the sensing layer comprises an analyte-responsive enzyme.

12. The method of claim 7, wherein the analyte-responsive enzyme is a glucose-responsive enzyme.

13. The analyte sensor of claim 1, wherein dexamethasone comprises from 0.1% to 25% (v/v) of a polymer layer formulation suitable for preparation of the polymer layer.

14. The method of claim 7, wherein dexamethasone comprises from 0.1% to 25% (v/v) of a polymer layer formulation suitable for preparation of the polymer layer.

15. The analyte sensor of claim 1, wherein the polymer layer comprising dexamethasone is disposed on the counter electrode.

16. The method of claim 7, wherein the polymer layer comprising dexamethasone is disposed on the counter electrode.

17. The analyte sensor of claim 1, wherein the polymer layer comprising dexamethasone is disposed on an exterior surface of the substrate.

18. The method of claim 7, wherein the polymer layer comprising dexamethasone is disposed on an exterior surface of the substrate.

19. The analyte sensor of claim 1, wherein the sensor has a sensitivity that is 90% or more of the initial sensitivity for 14 days or more following subcutaneous insertion of the sensor.

20. The analyte sensor of claim 1, wherein the sensor demonstrates a reduced early signal attenuation effect for 14 days or more following subcutaneous insertion of the sensor relative to the early signal attenuation effect observed in an otherwise identical sensor that does not include dexamethasone.

21. The analyte sensor of claim 1, wherein the sensor has a sensitivity that is 90% or more of the initial sensitivity for 14 days to 1 month following subcutaneous insertion of the sensor.

22. The analyte sensor of claim 1, wherein the sensor demonstrates a reduced early signal attenuation effect for 14 days to 1 month following subcutaneous insertion of the sensor relative to the early signal attenuation effect observed in an otherwise identical sensor that does not include dexamethasone.
